(19) United States
(12) Patent Application Publication
(10) Pub. No.: US 2024/0358558 A1
(43) Pub. Date: Oct. 31, 2024

(54) DURABLE ABSORBENT PANT HAVING A VISUAL SIGNAL

(71) Applicant: The Procter & Gamble Company, Cincinnati, OH (US)

(72) Inventors: Jill Marlene ORR, Liberty Township, OH (US); Jodi Lee BROWN, Cincinnati, OH (US); John Lee HAMMONS, Hamilton, OH (US); Grant Edward Anders STRIEMER, Fairfield Township, OH (US); J Michael BILLS, Mason, OH (US); Paul Thomas WEISMAN, Cincinnati, OH (US); Scott Kendyl STANLEY, Mason, OH (US); Cassandra Ann SEPELLO, Loveland, OH (US)

(21) Appl. No.: 18/645,742

(22) Filed: Apr. 25, 2024

Related U.S. Application Data
(60) Provisional application No. 63/498,934, filed on Apr. 28, 2023.

(51) Int. Cl. A61F 13/42; A61F 13/494; A61F 13/496

(52) U.S. Cl. CPC A61F 13/42; A61F 13/49406; A61F 13/496; A61F 2013/422; A61F 2013/428

(57) ABSTRACT

A durable absorbent pant (for example, underwear brief) to be worn for containing and absorbing discharges resulting from menstruation, where the pants include a visual signal to indicate the presence of a discharge or bodily exudate in one or more portions of the pant.

### CROSS REFERENCE TO RELATED APPLICATION

This application claims the benefit of U.S. Provisional Application No. 63/498,934, filed Apr. 28, 2023, the substance of which is incorporated herein by reference.

### FIELD OF THE INVENTION

This invention relates to features of durable absorbent pants (for example, underwear briefs) to be worn for containing and absorbing discharges resulting from menstruation, where the pants include a visual signal to indicate the presence of a discharge or bodily exudate in one or more portions of the pant.

### BACKGROUND

A variety of absorbent products, including disposable absorbent products as well as durable, reusable absorbent products, are commercially available for absorbing and retaining menstrual fluids and other vaginal discharges. In particular, the popularity of durable, reusable absorbent products, such as durable absorbent underwear and durable absorbent pads, is growing. Many users may prefer the fit and feel of durable, reusable absorbent products, which tend to feel more like underwear. As with other types of absorbent articles, users may also prefer for a durable absorbent product to have a clean and dry appearance, even after much of the absorbent capacity of the absorbent article has been used. Users may prefer that the top (user-facing) layer of the crotch portion of the underwear or pad, which is visible to the user when the underwear or pad is removed, conceals or disguises any stains that are present. For example, in some existing absorbent menstrual underwear products, the top layers of the crotch portions or gussets are dark-colored (e.g., black) to conceal menstrual stains.

The existing of durable, reusable absorbent products have a number of disadvantages. Some existing products may provide insufficient capacity and/or fluid handling performance. Even products that may provide sufficient capacity and/or fluid handling performance, may not effectively indicate the presence of menstrual fluid in the product and/or that the absorbent capacity of the product is being approached to enable the user to change the product before any leakage occurs. In particular, a durable, reusable absorbent product that has a dark-colored gusset for concealing menstrual stains may, albeit unintentionally, make it difficult for a user to identify the presence of menstrual fluid in the product and/or that the absorbent capacity of the product is being approached. As a consequence, the user may change the product prematurely, thereby underutilizing the capacity of the product, or belatedly, thereby exceeding the capacity of the product and potentially experiencing leaks.

Thus, a need exists for a durable absorbent product that provides an effective signal to a user that the absorbent capacity of the product is being approached, to enable the user to both utilize the entire capacity of the product and change the article before any leakage can occur.

### BRIEF DESCRIPTION OF THE DRAWINGS

FIG. 1 is a simplified depiction of an example of a brief pant, as it would appear laid out flat on a horizontal planar surface, front waist portion facing up.

FIG. 2 is a depiction of an example of the brief pant of FIG. 1, in an assembled but opened configuration wherein the front and rear waist portions have been separated at the hip portions or hip side seams, as it would appear laid out flat on a horizontal planar surface, wearer-facing surfaces facing up.

FIGS. 3A-3C are partial, schematic lateral cross section views of examples of absorbent gusset assemblies, through a side edge proximate to a leg opening.

FIGS. 4A and 4B are partial, schematic longitudinal cross section views of examples of pants including absorbent gusset assemblies, through a crotch portion and leg opening edge.

FIGS. 5, 6A-6C, 7A-7C, 8 and 9 are partial, schematic longitudinal cross section views of additional examples of pants including absorbent gusset assemblies, through a crotch portion and leg opening edge.

FIGS. 10A-10C are partial, schematic longitudinal cross section views of examples of pants including absorbent gusset assemblies, through an end of the gusset assembly.

FIG. 11 is a schematic vertical cross section, through a longitudinal center plane (as identified in FIG. 1) of one possible example of a pant with a gusset, oriented as it would be on a standing wearer.

FIG. 12A is a simplified depiction of an example of a brief pant, as it would appear laid out flat on a horizontal planar surface, front waist portion facing up.

FIG. 12B is a simplified depiction of an example of a shorts pant (or legged pant), as it would appear laid out flat on a horizontal planar surface, front waist portion facing up.

FIG. 13A is a plan view of an example of a crotch gusset laid out on a horizontal surface, wearer-facing surface facing the viewer.

FIG. 13B is a schematic vertical cross section of a portion of the gusset shown in FIG. 13A.

FIGS. 14-29 are plan views of examples of apertured wearer-facing surfaces.

### DEFINITIONS

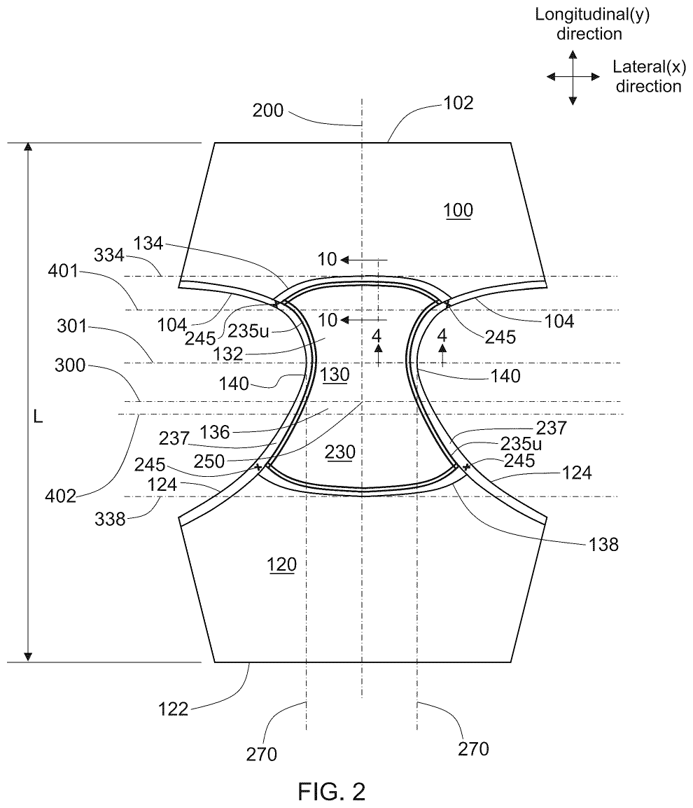

A “crotch gusset 230” is a structural component that includes at least two distinct layers including an absorbent layer and a liquid impermeable barrier layer, and bridges the front waist portion 100 and the rear waist portion 120. A crotch gusset has a “liquid impermeable barrier layer” if it exhibits z-Direction Leakage no greater than 0.1 ml of test fluid into a section of filter paper, in the Liquid Impermeability test described below. Referring to FIG. 2 by way of example, for purposes herein, crotch portion 130 is the portion of the pant, at a minimum, lying between crotch portion minimum front extent 401 and crotch portion minimum rear extent 402, longitudinally centered about crotch portion lateral axis 301 (which is drawn along the smallest width dimension measured between the crotch leg opening edges 140), plus 10 percent of the overall length L of the pant to the front and the rear of the crotch portion lateral axis 301. As suggested by FIG. 2, it may be desired that the crotch portion lateral axis 301 be disposed forward of the lateral axis 300 (which equally divides overall length L), rather than be co-located with lateral axis 300, for purposes of better fit about an adult female wearer's legs and lower torso. Thus, the respective boundaries between crotch portion 130 and front and rear waist portions 100, 120 for purposes herein are independent of the location(s) of any seams such as seams 134, 138 that may be present to join material(s) included in the crotch gusset 230 and material(s) included in the front and rear waist portions 100, 120.

With respect to a wearable garment such as an underwear pant, “durable” means that the garment is made predominantly of fabric that is knitted and/or woven from natural, semi-synthetic or synthetic fiber, thread or yarn, and may be normally laundered, or hand-washed and dried, for reuse/re-wear a plurality of times without substantial loss of original shape, structural integrity, absorbent function or other useful mechanical attributes that existed prior to laundering or washing.

As used herein, “fabric” means a web material that is knitted or woven of fibers, or threads or yarns of fibers. Herein, the term “fabric” does not include a film or a fibrous nonwoven web material.

As used herein, “film” means a skin- or membrane-like material that is cast, extruded or formed in place, from a molten thermoplastic material. Herein the term “film” does not include fibrous nonwoven web material or fabric.

With respect to a pant in an assembled but opened configuration, laid out flat on a horizontal planar surface, “longitudinal” refers to a direction generally perpendicular to a line tangent each of the left and right leg opening edges where they are closest the front waist edge. With respect to a pant in an assembled configuration, laid out flat on a horizontal planar surface, front waist portion facing up, “longitudinal” refers to a direction generally perpendicular to a line tangent each of the left and right leg opening edges where they are closest the front waist edge. “Length” refers to a dimension measured along the longitudinal direction. The longitudinal direction is deemed a “y-direction” in figures where indicated.

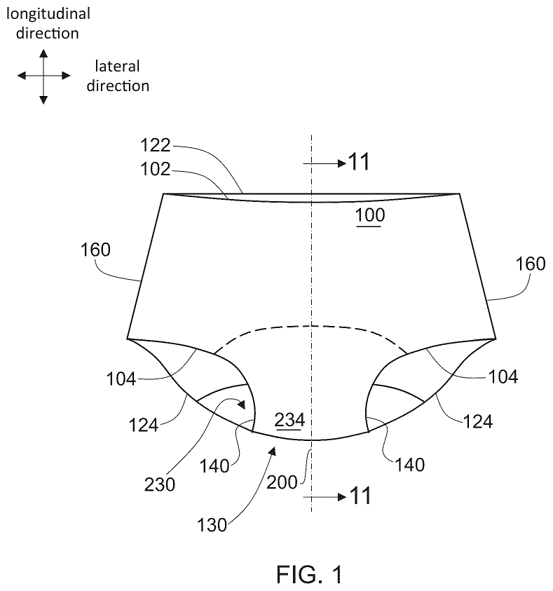

With respect to a pant in an assembled but opened configuration (e.g., as illustrated in FIG. 2), laid out flat on a horizontal planar surface, “lateral” refers to a direction generally parallel to a line tangent each of the left and right leg opening edges where they are closest the front waist edge. With respect to a pant in an assembled configuration (e.g., as illustrated in FIG. 1), laid out flat on a horizontal planar surface, front waist portion facing up, “lateral” refers to a direction generally parallel to a line tangent each of the left and right leg opening edges where they are closest the front waist edge. “Width” refers to a dimension measured along the lateral direction. The lateral direction is deemed an “x-direction” in figures where indicated.

With respect to a pant in an assembled but opened configuration (e.g., as illustrated in FIG. 2), laid out flat on a horizontal planar surface, the “z-direction” is the direction orthogonal to the longitudinal and lateral directions, i.e., the vertical direction relative the horizontal planar surface.

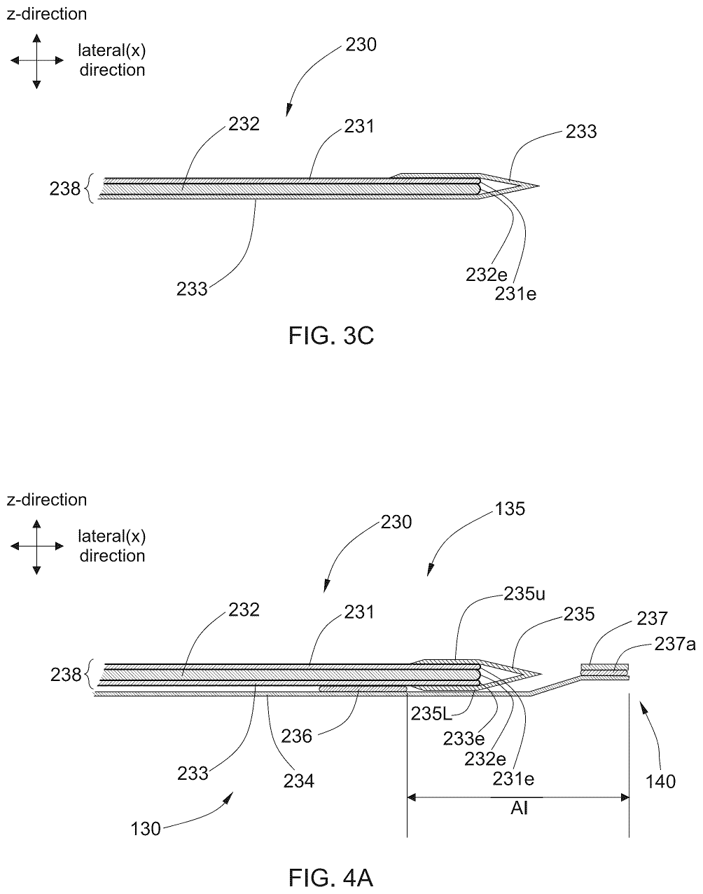

With respect to respective layer components in a crotch gusset of a pant in an assembled but opened configuration (e.g., as illustrated in FIG. 2), laid out flat on a horizontal planar surface, wearer-facing surfaces facing up, as between first and second layer components in the crotch portion, the terms “above,” “upper,” “superadjacent,” “below,” “lower,” “subjacent” and/or “beneath” describe the components' disposition along the z-direction relative each other. Thus, for example, referring to FIG. 4A, a wearer-facing layer 231 is disposed “above” an outward-facing layer 234, and conversely, the outward-facing layer 234 is disposed “below” the wearer-facing layer 231 and/or absorbent layer 232. “Superadjacent” and “subjacent” with respect to two layer components, mean further, that the two layer components are disposed in direct surface-to-surface contact with each other.

A yarn, thread, fiber, filament, web, film or fabric material, or a laminate or composite of any of these, is considered to be “elastically extensible” for purposes herein if, when a tensile force no greater than 50 gf/mm (gf per mm of sample width, where sample width is measured perpendicular to the stretch direction) is applied to the subject material along a stretch direction, the material may be extended along the direction to an elongated dimension of at least 130% of its original relaxed dimension (i.e., can extend at least 30%), without rupture or breakage which substantially damages the subject material; and when the force is removed from the subject material, the material retracts along the stretch direction to recover at least 40% of such elongation. To illustrate, if a section of fabric having an original relaxed length of 100 mm and a sample width of 40 mm can be elongated by tensile force of 2000 gf (50 gf/mm) in a direction along its length to 130 mm length without substantial damage, and will retract upon removal of the force to a length no greater than 118 mm (130 mm−118 mm=12 mm=40% of 30 mm), it is “elastically extensible” as defined herein. “Elongation,” used herein to quantify and express an amount of strain imparted to an elastically extensible material in a stretch direction, means: {[(strained length of the strand)−(length of the strand prior to straining)]/(length of the strand prior to straining)}, ×100%. For a monolithic material such as a cast or extruded film material, “elastomeric” means “elastically extensible” as defined herein.

With respect to two opposing surfaces of a layer component of a pant, “wearer-facing” refers to the surface that faces the wearer's skin when the pant is worn normally; and “outward-facing” refers to the surface that faces away from the wearer's skin. With respect to two distinct layered components of a pant, the “wearer-facing” component is the component that is disposed closest the wearer's skin when the pant is worn normally; and the “outward-facing” component is the component that is disposed farthest from the wearer's skin.

For purposes herein, “pant” includes any garment adapted for wear about the human lower torso, including a front waist portion and a rear waist portion that join about the wearer's hips and beneath the wearer's crotch, to form a garment having a waist opening and a pair of leg openings. Herein, the term “pant” encompasses (but is not limited to) a garment defined herein as a “brief pant”; a garment defined herein as a “legged pant”, and any other garment whether adapted for use as underwear or outerwear, having such features.

For purposes herein, unless otherwise specified, with respect to the proportionate content of a component material in a combination or structure, “predominant” means the component constitutes the majority of the weight of the combination or structure.

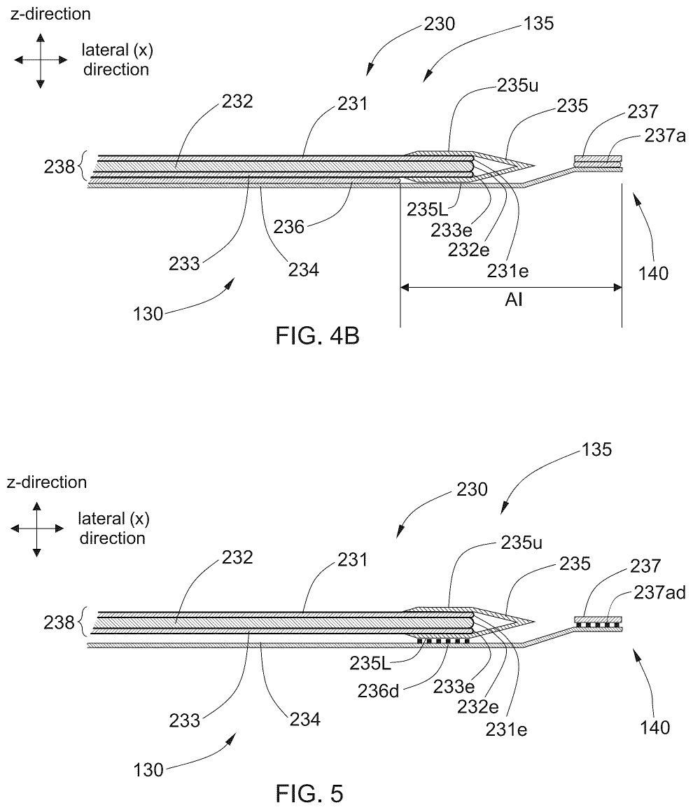

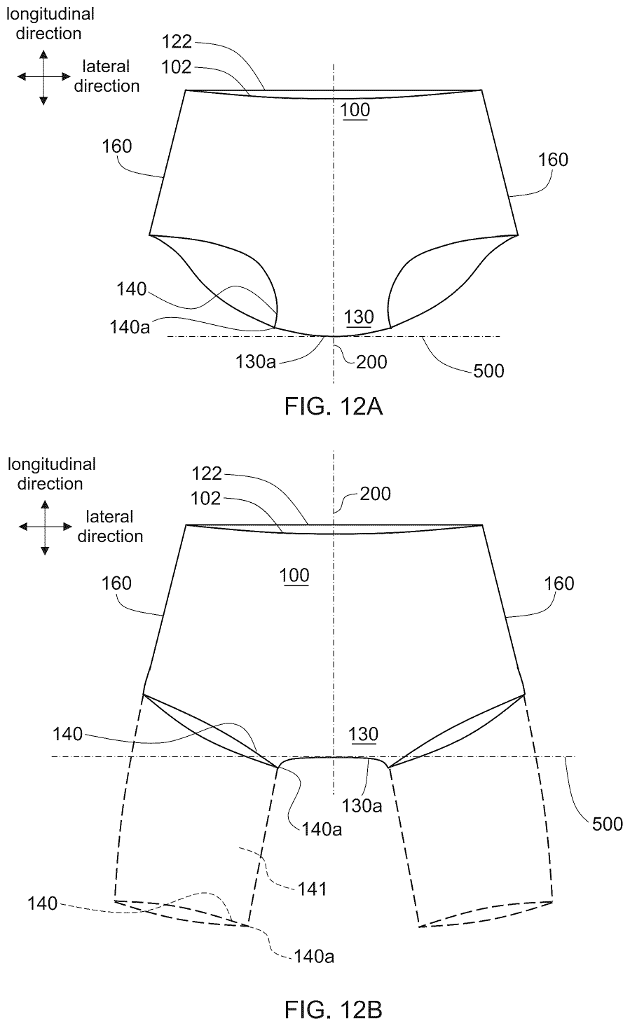

For purposes herein, a “brief pant” is distinguished from a “legged pant” by the configuration of the inside leg edges, resulting from the manner in which the component materials are shaped, sized, proportioned and seamed or otherwise affixed together. FIG. 12A depicts an example of a brief pant and FIG. 12B depicts an example of a legged pant in alternative forms. When the garment in its assembled condition is laid out flat on a horizontal planar surface, front waist portion facing up, and a lateral crotch tangent line 500 is drawn perpendicularly to longitudinal axis 200 and tangent to the point at which the crotch portion lower profile 130a intersects the longitudinal axis 200,
for a brief pant, the lowermost points 140a along the inside leg opening edges 140 are disposed along or above the crotch tangent line 500 (i.e., toward the front waist edge 102) (see FIG. 12A); and
for a legged pant, the lowermost points 140a along the inside leg opening edges 140 are disposed below the crotch tangent line 500 (i.e., away from the front waist edge 102) (see FIG. 5B).
   As reflected in FIG. 12B, for purposes herein, a legged pant may have leg portions 141 of any length, wherein the lowermost points 140a of inside leg opening edges 140 are disposed below the crotch tangent line 500. Underwear shorts pants such as “boy short” styles will generally have shorter leg portions as suggested in the solid-line portions of FIG. 12B, while other legged pants may have leg portions of varying lengths, as suggested by the dashed-line portions of FIG. 12B.

### DESCRIPTION

As noted in the Background, a problem presented in existing of durable, reusable absorbent products is that such products do not effectively indicate the presence of menstrual fluid in the product and/or that the absorbent capacity of the product is being approached. In particular, a durable, reusable absorbent product that has a dark-colored gusset for concealing menstrual stains may make it challenging for a user to identify the presence of menstrual fluid in the product and/or that the absorbent capacity of the product is being approached. As a consequence, the user may change the product prematurely, thereby underutilizing the capacity of the product, or belatedly, thereby exceeding the capacity of the product and potentially experiencing leaks. It is believed, however, that a durable absorbent product that provides an effective signal to a user that menstrual fluid is present in the product and/or that the absorbent capacity of the product is being approached, would enable the user to better utilize the entire capacity of the product and to avoid leakage.

Currently marketed durable absorbent products, particularly durable absorbent pants for adults, do not effectively communicate to the user the presence of menstrual fluid in the product and/or that the absorbent capacity of the product is being approached. In particular, some currently marketed products, such as durable absorbent menstrual underwear, have dark-colored gussets for concealing menstrual stains. These dark-colored gussets may make it difficult for a user to identify the presence of menstrual fluid in the product and/or that the absorbent capacity of the product is being approached. It is believed that the durable absorbent product industry to date has failed to recognize the importance of providing an easily identifiable signal or indicator of the presence of menstrual fluid in the product and/or that the absorbent capacity of the product is being approached. The industry has also not recognized that a durable absorbent product that conceals or disguises menstrual stains, while also providing an easily identifiable signal or indicator of the presence of menstrual fluid in the product and/or that the absorbent capacity of the product is being approached may be designed.

The durable absorbent products of the present disclosure include easily identifiable signals or indicators of the presence of menstrual fluid in the product and/or that the absorbent capacity of the product is being approached.

### Signal/Indicator

A signal or indicator that communicates the presence of menstrual fluid in a durable absorbent product and/or that the absorbent capacity of the durable absorbent product is being approached may be visual. A signal or indicator is considered visual if it can indicate the presence of a menstrual fluid by its visual state. A visual indicator may be configured to change from an initial visual state to a subsequent visual state, when indicating the presence of a liquid bodily exudate, such as menstrual fluid. Throughout the present disclosure, the term visual state refers to an appearance that can be perceived by an unaided human with normal vision in standard lighting conditions. A visual state may comprise one or more colors, variations of color(s), patterns, letters, numbers, symbol, designs, images, and/or other visual devices. Colors include well known colors, such as red, orange, yellow, green, blue, and purple. Variations of a color include variations in chroma, hue, and brightness, among others. While these informal terms are used for ease of reference, embodiments of the present disclosure are intended to encompass all colors which can be perceived by an unaided human with normal vision in standard lighting conditions. Also, throughout the present disclosure, unless otherwise stated, the presence of menstrual fluid refers to the presence of a concentration of the menstrual fluid that is sufficient to cause a visual signal or indicator to change visual states.

A signal or indicator that can indicate the absorbent capacity of the durable absorbent product is being approached may be referred to herein as a fullness signal or indicator. Such fullness signals may indicate the progression or movement of menstrual fluid in the absorbent product. For example, a visual fullness signal may include one or more visual indicating areas-two-dimensional regions configured to indicate the presence of menstrual fluid by their visual state. A visual fullness signal may include a series of indicating areas or a pattern of indicating areas. A visual fullness signal may include a first indicating area and a second indicating area, where the first indicating area changes visual states before the second indicating area changes visual states.

The visual signal or indicator may comprise a colorant adapted to change its appearance, such as appear, disappear, or change color, upon contact with menstrual fluid. For example, the visual signal or indicator may comprise a hydrochromic ink, which changes color when exposed to moisture, e.g., menstrual fluid. Water-based and solvent-based hydrochromic inks and coatings have previously been described and used on textiles and fabrics. A hydrochromic ink that changes color from yellow to blue, when exposed to moisture, has been described. The hydrochromic ink may be reversible. For example, reversible hydrochromic inks that are white when dry, transparent when exposed to moisture, and then, upon drying, revert back to white have been described. Alternatively or additionally, a change in reflectivity may provide a visual signal.

A hydrochromic ink may be applied to the durable absorbent product described herein in a number of ways. For example, a hydrochromic ink may be applied by using a template over a completed crotch gusset, before the crotch gusset is attached to the body fabric of the durable absorbent product. A hydrochromic ink may be applied via screen printing (flatbed, rotary, cylinder), flexographic printing, gravure printing, lithographic printing, roller coating, and/or ink jet printing. In the context of the durable absorbent products disclosed herein, the hydrochromic ink is preferably able to withstand multiple launderings of the product.

The visual fullness indicator or signal may comprise one or more apertures through a wearer-facing layer of the durable absorbent product, as described in detail below.

### Durable Absorbent Pant

Durable absorbent products include absorbent brief pants (i.e., a pant with a crotch portion including an absorbent structure and a liquid impermeable barrier layer to the outside thereof). For underwear, many women prefer brief pants rather than legged pants such as “boy shorts” styles for ordinary daily wear. This is due to issues of comfort; unlike a legged, shorts-type pant, a brief pant ordinarily will not ride up and bunch about the legs from changes of body position, and thereby be a source of unwanted concentration of material bulk, tightness about the legs or other discomfort under outer clothing. Further, due to the manner in which their leg edges tend to cause the pant to fit through the crotch region of the body, brief pants having suitable elastic stretch characteristics may be preferred for maintaining a close fit about the female genital/urethra area, for purposes of protecting against leakage of menstrual fluid.

Referring to FIGS. 1 and 2 as an illustrative but non-limiting example, a pant may include a front waist portion 100, a rear waist portion 120 and a crotch portion 130 bridging the front and rear waist portions. Front waist portion 100 has a front waist edge 102, and left and right front leg opening edges 104. Rear waist portion 120 has a rear waist edge 122, and left and right rear leg opening edges 124. Crotch portion 130 has left and right crotch leg opening edges 140. Crotch portion 130 may include the entirety or a portion of a crotch gusset 230 that may include several layer components that will be described further below. (For purposes herein, the “crotch portion 130” is a portion of a pant identified as described herein, and its forward and rearward extents are independent of specific components or structures.) Herein, a “gusset” 230 is a structural component that includes at least two distinct layers including an absorbent layer and a liquid impermeable barrier layer, and is disposed in the crotch portion and may bridge the front waist portion 100 and the rear waist portion 120. A gusset has a “liquid impermeable barrier layer” if it exhibits z-Direction Leakage no greater than 0.1 ml of test fluid into a section of filter paper, in the Liquid Impermeability test described below. Referring to FIG. 2 by way of example, for purposes herein, crotch portion 130 is the portion of the pant, at a minimum, lying between crotch portion minimum front extent 401 and crotch portion minimum rear extent 402. Crotch portion 130 is longitudinally centered about crotch portion lateral axis 301 (which is drawn along the smallest width dimension measured between the crotch leg opening edges 140), and includes 10 percent of the overall length L of the pant to the front and the rear of the crotch portion lateral axis 301. As suggested by FIG. 2, it may be desired that the crotch portion lateral axis 301 be disposed forward of the lateral axis 300 (which equally divides overall length L), rather than be co-located with lateral axis 300, for purposes of better fit about an adult female wearer's legs and lower torso. Thus, the respective boundaries 401, 402 between crotch portion 130 and front and rear waist portions 100, 120 for purposes herein, are independent of the location(s) of any seams such as seams 134, 138 that may be present to join material(s) included in the crotch gusset 230 and material(s) included in the front and rear waist portions 100, 120. Material forming the front and rear waist portions 100, 120 may be integral and continuous across, or may be joined at seams, at or proximate hip portions 160, to complete a pant structure having a waist opening and a pair of leg openings.

Material(s) forming one or both of forward and rearward portions 132, 136 of crotch portion 130 may be integral and continuous with material(s) forming front and rear waist portions 100, 120, or alternatively, one or both of forward and rearward portions 132, 136 of crotch portion 130 and/or crotch gusset 230 may be substantially formed of one or more sections or layers of material that are distinct from material(s) substantially forming one or both of front and rear waist portions 100, 120, and crotch gusset 230 may be joined to front and rear waist portions 100, 120 at one or both of forward seam 134 and rearward seam 138. In illustrative but non-limiting examples suggested in FIGS. 1, 2 and 10A-10C, front waist portion 100, an outward-facing layer 234 of crotch portion 130, and rear waist portion 120, may all be formed partially or entirely of a first single, continuous section of material, e.g., the fabric from which the main body of the pant is formed. As reflected in the examples shown in FIGS. 4A, 4B, 5, 6A-6C, 7A-7C, 8, 9 and 10A-10C, one or more additional layers of material 231, 232, 233 may be disposed over the wearer-facing side of outward-facing layer 234 in crotch portion 130 as shown, and be connected or affixed directly or indirectly to layer 234 via, e.g., stitching/sewing, adhesive bonding, thermal bonding (fusing or welding) or other suitable attachment/joining mechanism (hereinafter, attachment mechanism) at, e.g., forward and rearward gusset seams 134, 138 and/or along the crotch leg opening edges] 140.

In other non-limiting examples (not specifically shown), the sections of materials respectively forming front waist portion 100, rear waist portion 120 and crotch portion 130 may be entirely separate and distinct, and joined via any suitable attachment mechanism at, e.g., forward and rearward seams 134, 138. This configuration may be preferred in some circumstances because it may provide the designer with greater flexibility in selection of the respective materials for the waist and crotch portions with respect to appearance, feel, weight, breathability, elongation, stretch characteristics and cost.

### Crotch Portion/Crotch Gusset

### Gusset Components

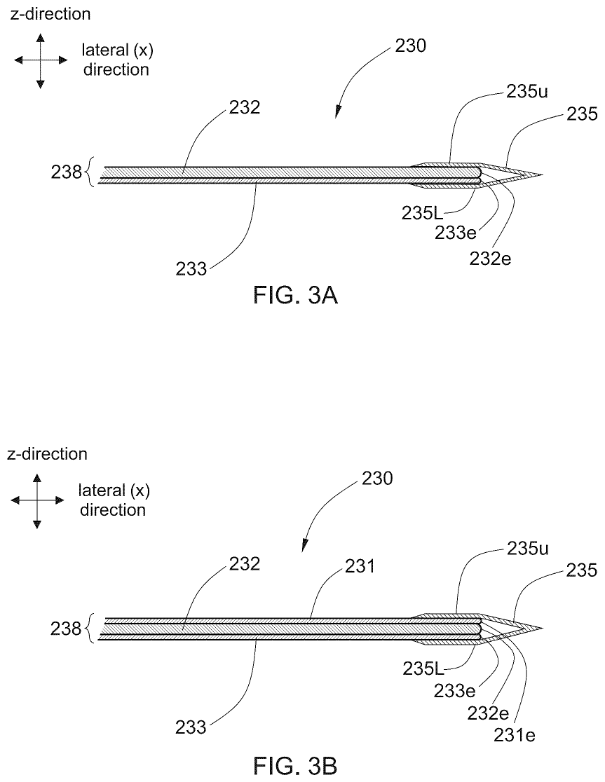

Referring to FIGS. 3A, 3B and 3C, a crotch gusset 230 includes components of an absorbent assembly 238 which include, at a minimum, an absorbent layer 232 and a barrier layer 233. The absorbent layer 232 may be one or more layers of suitable fabric, selected for desired absorbency and stretch characteristics. Absorbent layer 232 will be underlaid by a barrier layer 233, which may be a suitable polymeric film, or alternatively, a fabric that is effectively resistant to passage of liquid therethrough under ordinary conditions of wear of garments of the type described herein. Barrier layer 233 serves to prevent liquids absorbed by the absorbent layer 232 from passing by wicking or capillary action out to an outward-facing layer of the pant, and from there out to outer clothing, bedclothes, etc.

In some examples (not specifically shown), absorbent layer 232 may include or be formed of two, three or more distinct sublayers of material disposed superadjacent/subjacent one another. The respective sublayers may have different sizes, dimensions and/or surface areas along the x- and y-directions, and may be formed of or include respectively differing materials. In some examples, one or more of the sublayers may be formed of or include a nonwoven web material. In such examples, for reasons explained herein, it may be preferred that such sublayers be constituted and manufactured so as to possess elastic extensibility, or alternatively, be arranged within the gusset so as to be unbonded/unattached to superadjacent or subjacent layers (e.g., elastically extensible layers), over a majority of the overlapping surface area therebetween, to avoid impeding elastic stretch of such superadjacent/subjacent layers.

Barrier layer 233 may be formed of or include a suitable polymeric film. The polymer component(s) of the film may be selected so as to impart one or both of effective liquid impermeability and elastic extensibility thereto. In some examples, the film may be adapted to be liquid impermeable under ordinary conditions of use contemplated herein, but be effectively gas and/or vapor permeable (i.e., “breathable”), so as to allow water vapor to more easily escape the garment, and thereby improve comfort and reduce chances for overhydration of the wearer's skin (which can cause or contribute to undesirable growth of microorganisms, skin irritation, rash, etc.). In some examples, it may be preferred that barrier layer 233 be unattached to overlying absorbent layer(s) in middle/central regions of the gusset between peripheral edge seams, to reduce binding, stiffening and elasticity-reducing effects of bonding or affixing superadjacent/subjacent layers.

In some examples crotch gusset 230 may include an additional, overlying wearer-facing layer 231. Wearer-facing layer 231 may be formed of a suitable fabric that has a comfortable feel against the skin while being configured to function to conduct liquids down to the absorbent layer(s) 232 following a discharge, and subsequently, to isolate the absorbed fluids in the absorbent layer from the wearer's skin, to help impart a relatively dry feel to the pant for the wearer, even after a discharge.

The wearer-facing layer of the crotch gusset may have an initial L* value of less than about 60, or less than about 40, measured using the CIELAB color scale. The wearer-facing surface of the absorbent layer may have an initial L* value of greater than about 50, or greater than about 80, measured using the CIELAB color scale. The CIELAB color scale is described in detail below.

The wearer-facing layer may have an opacity of greater than about 20, or greater than about 25, or greater than about 50. The opacity of a material is the degree to which light is blocked by that material. A higher opacity value indicates a higher degree of light block by the material.

In most designs it may be preferred to provide edge sealing of the absorbent layer 232 and wearer-facing layer 231 (if included), about the perimeters of these layers, e.g., about a portion or the entirety of the perimeter of the gusset 230, or at least at the perimeter edges of the gusset proximate the leg opening edges 140 in the crotch portion 130 of the pant. Since these layers may be formed of fabrics configured to accept and transfer liquid via wicking or capillary action, it may be desired to block liquid movement out the perimeter edges of these layers, which can result in leakage and soiling of outward-facing layer 234 of the crotch portion, outer clothing, bedclothes, etc.

Referring to FIGS. 3A and 3B, in some examples, edge sealing may be provided by including an edge sealing strip 235. Edge sealing strip may formed of a film or fabric that is suitably waterproof or effectively resistant to passage of liquid therethrough, under conditions of use contemplated herein. Edge sealing strip 235 is preferably sealingly adhered to barrier layer 233 so as to be effectively unitary therewith, and wraps up and around the perimeter edges of the absorbent layer(s) 232 and wearer-facing layer 231 (if included), and is affixed to the wearer-facing surface of the uppermost layer proximate the perimeter thereof. So configured, edge sealing strip can be effective to block passage of liquid that may wick to the perimeter edges of these layers, out therefrom. Edge sealing strip 235 may be formed of an inherently tacky material having sufficient adhesive properties to enable it to adhere to barrier layer 233 and wearer-facing layer 231 with or without application of heat and pressure, or alternatively, edge sealing strip 235 may be adhered to these component via a suitable adhesive. For purposes of retaining any inherent elastic stretchability of individual layers 231, 232, 233 in the assembly, it may be desired that edge sealing strip 235 be formed of an elastomeric material. In some examples, edge sealing strip 235 may be formed of a substantially liquid impermeable polymeric film having partially or predominantly a silicone (polysiloxane) component, which may be preferred for its relative softness, pliability, elasticity/elastic extensibility, durability and resilience.

In other examples such as suggested in FIG. 3C, edge sealing may be provided by an integral, continuous extension of barrier layer 233, providing a perimeter margin portion thereof of a size sufficient to wrap up and around the perimeter edges of the absorbent layer(s) 232 and wearer-facing layer 231 (if included), and be affixed via adhesive or other suitable mechanism to the wearer-facing surface of the uppermost layer proximate the perimeter thereof. So configured, such wrapping margin portion(s) of barrier layer 233 can be effective to block passage of liquid that may wick to the perimeter edges of the absorbent layer(s) 232 and wearer-facing layer 231 (if included), out therefrom.

The width of the crotch portion is preferably sufficient for providing effective coverage and contact against the body through a variety of normal body positions and movements. Accordingly, it may be desired that the crotch portion 130 have a minimum width or minimum crotch width (measured along crotch portion lateral axis 301, which is drawn along the smallest width dimension measured between the crotch leg opening edges 140) of at least about 5.0 cm, or at least about 6.5 cm, or at least about 7.0 cm, or at least about 8.0 cm.

### Aperture

The durable absorbent pant may include a visual fullness indicator or signal. Preferably, the visual fullness indicator or signal comprises one or more apertures through the wearer-facing layer. The durable absorbent pant, may comprise: a front waist portion with a front waist edge and left and right front leg opening edges; a rear waist portion with a rear waist edge and left and right rear leg opening edges; a crotch portion comprising a crotch gusset, the crotch portion having a forward portion meeting the front waist portion and a rearward portion meeting the rear waist portion; and left and right hip side portions joining the front waist portion to the rear waist portion and thereby forming a waist opening with a waist opening edge comprising the front waist edge and the rear waist edge, and left and right leg openings; where, when the pant is in an opened configuration in which the front waist portion and rear waist portion are separated at the hip side portions, the pant has a longitudinal axis and a lateral axis, with an intersection thereof, the intersection occurring in the crotch portion; where each of the front waist portion, rear waist portion and crotch gusset comprises a knitted material; and where the crotch gusset comprises a wearer-facing layer, an absorbent layer having a length measured along the longitudinal axis, and a liquid impermeable barrier layer disposed to the outward-facing side of the absorbent layer; and where the wearer-facing layer comprises one or more apertures, where the absorbent layer is visible through the one or more apertures. The one or more apertures may visibly change appearance from an initial visual state to a subsequent visual state corresponding, respectively, with absence and presence of a liquid bodily exudate in the absorbent layer or a portion thereof.

The one or more apertures may have a circumscribing perimeter edge having a point location therealong, the point location being disposed a distance that is greater than about 10%, or greater than about 20% of the length of the absorbent layer longitudinally away from a crotch portion lateral axis. The one or more apertures may have a circumscribing perimeter edge having a point location therealong, the point location being disposed at about the longitudinal axis. The one or more apertures may have a circumscribing perimeter edge having a point location therealong, the point location being disposed a distance that is less than about 50% of a minimum width of the crotch portion laterally away from the longitudinal axis.

A visible change in the appearance of the one or more apertures, from an initial visual state to a subsequent visual state (corresponding, respectively, with absence and presence of a liquid bodily exudate in the absorbent layer or a portion thereof) is measured using the CIELAB color scale by the Color Measurement Method included herein. The CIELAB color scale may also be referred to as L*a*b*, a color scale defined by the International Commission on Illumination (abbreviated CIE) in 1976. The CIELAB color space is a device-independent, 3D color space that enables accurate measurement and comparison of all perceivable colors using three color values. The letters L*, a* and b* represent each of the three values the CIELAB color space uses to measure objective color and calculate color differences. L* represents lightness from black to white on a scale of zero to 100, while a* and b* represent chromaticity with no specific numeric limits. Negative a* corresponds with green, positive a* corresponds with red, negative b* corresponds with blue and positive b* corresponds with yellow.

The wearer-facing layer of the crotch gusset may have an initial L* value of less than about 60, or less than about 40. The wearer-facing surface of the absorbent layer may have an initial L* value of greater than about 50, or greater than about 80. A ΔL between an initial L* value of the wearer-facing layer and an initial L* value of a wearer-facing surface of the absorbent layer may be greater than about 5, or greater than about 10. A ΔE between an initial L*a*b* color value of the wearer-facing layer of the crotch gusset and a subsequent L*a*b* color value of the wearer-facing layer of the crotch gusset may be less than about 30, or about 0.1 to about 20, or about 0.5 to about 10. A ΔE between an initial L*a*b* color value of the wearer-facing surface of the absorbent layer and a subsequent L*a*b* color value of the wearer-facing surface of the absorbent layer may be greater than about 10, or greater than about 50. A ΔE_{initial-subsequent} of the wearer-facing surface of the absorbent layer may be greater than a ΔE_{initial-subsequent} of the wearer-facing layer of the crotch gusset. A ΔE between a subsequent L*a*b* color value of the wearer-facing layer and a subsequent L*a*b* color value of a wearer-facing surface of the absorbent layer may be greater than about 10, or greater than about 20.

The wearer-facing layer may have an opacity of greater than about 20, or greater than about 25, or greater than about 50. The opacity of a material is the degree to which light is blocked by that material. A higher opacity value indicates a higher degree of light block by the material.

The shape of each of the one or more apertures in the wearer-facing layer of the crotch gusset may be a circle, an hourglass, a rectangle, a square, a diamond, a rounded diamond, a football, an oval, a clover, a heart, a triangle, a tear-drop, an ellipse, or a combination thereof, preferably a circle. The dimensions of the one or more apertures may also be selected to optimize the visual fullness signal. The one or more apertures may together form a pattern that is symmetric about the longitudinal axis, symmetric about the lateral axis, or symmetric about both the longitudinal and lateral axes. The one or more apertures may together form a pattern that is asymmetric about the longitudinal axis, asymmetric about the lateral axis, or asymmetric about both the longitudinal and lateral axes.

The length of the one or more apertures may be about 1 mm to about 20 mm, about 1 mm to about 15 mm, or about 1 mm to about 10 mm. The area of the one or more apertures may be about 1 mm² to about 150 mm², about 1 mm² to about 100 mm², about 1 mm² to about 90 mm², about 1 mm² to about 80 mm², about 1 mm² to about 70 mm², about 1 mm² to about 60 mm², about 1 mm² to about 50 mm², about 1 mm² to about 40 mm², about 1 mm² to about 30 mm², or about 1 mm² to about 20 mm². The perimeter of the one or more apertures may be about 1 mm to about 50 mm, about 1 mm to about 40 mm, about 1 mm to about 30 mm, or about 1 mm to about 20 mm. The width of the one or more apertures may be about 1 mm to about 20 mm, or about 1 mm to about 15 mm, or about 1 mm to about 10 mm. The percentage of the wearer-facing layer that is apertured, also referred to herein as the percentage of open area, may be less than about 25%, or less than about 20%, or less than about 15%. The percentage of open area may be from about 0.1% to about 10%, or about 0.2% to about 8%, or about 0.5% to about 5%.

The wearer-facing layer of the crotch gusset may have fewer than 5 apertures/cm², or fewer than 4 apertures/cm², or fewer than 3 apertures/cm², or fewer than 2 apertures/cm². The wearer-facing layer of the crotch gusset may have less than 60 total apertures, or less than 40 total apertures, or less than 30 total apertures. When the wearer-facing layer comprises more than one aperture, e.g., a plurality of apertures, the apertures may be spaced uniformly or non-uniformly (at varying distances) from one another. Additionally, a plurality of apertures may be arranged in groups or patterns, where the groups or patterns are uniformly spaced or non-uniformly spaced apart.

### Caliper

For purposes of minimized bulk, it may be desired that the combination of layered materials present in the crotch portion 130 and crotch gusset 230 be selected and configured so has to have a central z-direction Caliper not exceeding about 6 mm, or not exceeding about 5.5 mm, and or not exceeding about 5 mm. (Herein, the “central z-direction Caliper” or “central Caliper” is measured at a location in the crotch portion 130 corresponding with the intersection 250 of the longitudinal 200 and lateral 300 axes of the pant (shown in FIG. 2), when opened and laid out flat on a horizontal planar surface.) Based on disclosure herein and teachings available in the art concerning textiles, persons of ordinary skill in the art will be equipped to select a combination of suitable materials to create a structure in the crotch portion 130 having such Caliper, along with other features and attributes described herein.

### Absorption Capacity

In order for a durable absorbent pant to provide suitable absorbency and protection against leakage of menstrual fluid over a reasonable duration of wear, for a woman experiencing menstruation, while balancing concerns for limiting caliper/bulkiness of the crotch portion while limiting the required relative planar size/surface area of an absorbent portion, it may be desired that a combination of material forming the structure within the crotch portion 130 of the pant have an Area Absorption Capacity from 0.1 ml/cm² to 0.4 ml/cm², and a Volume Absorption Capacity from 0.4 ml/cm³ to 1.0 ml/cm³, measured according to the Absorption Capacity Measurement method set forth in the publication of U.S. App. Ser. No. 63/397,523. Based on disclosure herein and teachings available in the art concerning textiles, persons of ordinary skill in the art will be equipped to select a combination of suitable materials to create a structure in the crotch portion 130 having such absorption capacity.

### Elastic Stretch Attributes

Through experimentation it has been learned that imparting a pant with appropriate directional elastic stretch characteristics within the crotch portion 130 are important to provide a pant with a crotch portion that is both comfortably and securely held against the wearer's body surfaces, in position to intercept and absorb an adhering flow of menstrual fluid, and thereby prevent leakage from the pant, through normal ranges of body positions and movements. It has been learned that the combination of materials present in the crotch portion 130 preferably should exhibit a maximum Longitudinal Elongation of 25 percent to 100 percent, measured according to the Maximum Elongation Measurement Method set forth in the publication of U.S. App. Ser. No. 63/397,523. Alternatively, or additionally, it may be desired that the combination of materials present in the crotch portion 130 exhibit a Longitudinal Tensile Modulus, measured according to the Longitudinal Tensile Modulus Method set forth in the publication of U.S. App. Ser. No. 63/397,523 (reflecting the presence of one or more materials that impart elasticity) of 10 gf/mm to 100 gf/mm. Based on disclosure herein and teachings available in the art concerning textiles, persons of ordinary skill in the art will be equipped to select a combination of suitable materials to create a structure in the crotch portion 130 having such longitudinal elongation and tensile modulus properties.

### Materials Selection

Generally, the front and rear waist portions 100, 120 of suitable examples of a pant may be formed of any fabric material or combination of fabric and other materials known and used as components of underwear, swimwear or athletic/active wear, exhibiting suitable attributes that may include, depending upon the location of the fabric within the structure, pleasing feel against the skin (softness and/or low-friction/smooth/silky feel), low caliper/bulk, elongation capability, clastic extensibility, absorbency, wicking ability, breathability, etc. In one example, the front and rear waist portions 100, 120, and the outward-facing layer 234 of the crotch gusset 230, may include or be formed of a knitted stretch fabric, in some examples, a combination of one or more of nylon, polyester, cotton and elastane fibers (e.g., LYCRA SPANDEX (a product of The Lycra Company, Wilmington, DE)).

Additional layer components of crotch portion 130 and/or crotch gusset 230, as herein described requiring absorbency and longitudinal elastic stretch attributes, may include a combination of several materials selected to impart the structure with the desired attributes.

### Fabric Structure

Most durable fabrics exhibit anisotropic elongation capabilities.

Woven fabrics, formed by weaving, are formed of two groups (warp group and weft group) of interlaced constituent yarns or threads, the yarns or threads within each group being substantially parallel to each other, and substantially perpendicular to the yarns or threads in the other group, along the plane of the fabric. Unless the constituent yarns or threads are themselves formed of extensible material, woven materials have relatively low elongation capabilities along the warp and weft directions, and have relatively higher elongation capabilities along the two bias directions approximately 45 degrees from the warp and weft directions. Consequently, where it is desired for particular reasons that a layer component of the crotch portion 130 and/or crotch gusset 230 be a woven material, it may be desired that the material be oriented within the crotch portion such that one of the warp and weft directions is oriented from approximately 30 degrees to approximately 60 degrees, preferably from approximately 38 degrees to approximately 52 degrees, and more preferably approximately 45 degrees from the longitudinal direction of the pant, so as to provide maximum available longitudinal elongation capability for the woven layer.

However, when stretched along the bias direction, woven fabrics typically exhibit a substantial Poisson effect contraction along the trans-stretch direction (90 degrees from the stretch direction). When such a fabric is included in the crotch portion 130 with its bias oriented approximately along the longitudinal direction of the pant, the Poisson contraction effect may cause the crotch portion to laterally narrow, which may be deemed undesirable when effective coverage of the wearer's body through the crotch region is desired.

The constituent yarns or threads of knitted fabrics, by contrast, do not follow straight paths along the plane of the fabric, and are neither parallel nor perpendicular to each other. Rather, each constituent yarn or thread of a knitted fabric follows a looping path along successive rows, interlooping with one or more constituent yarns or threads in adjacent rows. As a consequence, knitted fabrics exhibit relatively greater elongation capability along all directions as compared with woven fabrics, even where the constituent yarns or threads themselves are not extensible. For this reason, unless a woven fabric is desired for a particular reason, it may be preferred that a knitted fabric be used to form any one or more, or all, of the fabric layers present in the crotch portion 130 and crotch gusset 230 of the pant.

Even so, most types of knitted fabrics have elongation capabilities that are anisotropic along the plane of the fabric, having a first direction of greatest elongation capability and a second direction, perpendicular to the first direction, of least elongation capability. Accordingly, when knitted fabric is selected and used to form one or more layers present in the crotch portion 130 and crotch gusset 230 of the pant, it may be desired that the fabric(s) forming any, some or all of the layers be oriented such that their directions of greatest elongation capability are at least approximately parallel with the longitudinal direction of the pant.

In some circumstances it may be desired that a knitted fabric selected to form a layer be a rib knit type. Rib knitted fabrics exhibit relatively high elongation capability along a direction parallel to the knit rows (perpendicular to the “ribs”), with relatively low Poisson contraction effect along the trans-stretch direction. Thus, in some circumstances, it may be desired that one or more layers present in the crotch portion 130 and crotch gusset 230 of the pant be formed of a rib knit fabric, with the “ribs” oriented substantially along the lateral direction of the pant.

In some circumstances it may be desired that the absorbent layer 232 be formed of either woven or knitted terrycloth, for purposes of increasing aggregate fiber surface area and capillarity per unit fabric surface area, and thereby, providing increased absorbency to the absorbent layer 232, while still providing a durable fabric, in contrast to a nonwoven batt or matt of fibers. In conjunction therewith or as an alternative, and for purposes of enhancing absorbency, it may be desired that constituent fibers of the yarn(s) or thread(s) from which the absorbent layer fabric material is knitted or woven be in the form of microfibers (i.e., fibers having an average denier of one (1) or less). It may be further desired that the constituent fibers be split microfibers. Yarns or threads formed of microfibers, particularly split microfibers, provide relatively greater fiber surface area per unit yarn/thread denier. When the fiber surfaces are hydrophilic, this imparts relatively greater absorbency to the fabric.

### Fabrics Constituent Yarn/Thread Compositions

As contemplated herein, wearer-facing 231 and absorbent 232 layers present in the crotch portion and crotch gusset of the pant are expected to be exposed to discharges of menstrual fluid, and are expected to receive, absorb and retain these fluids for a reasonable duration of wear time, preferably while leaving the wearer-facing surfaces as dry-feeling as possible. It may be desired that a wearer-facing layer 231 have a soft feel against the skin.

Accordingly, it may be desired that the wearer-facing layer be formed of a material that has a soft feel and has suitable wicking attributes so as to efficiently conduct discharged liquid to an absorbent layer beneath, while having minimized tendency to retain liquid and thereby have a wet feel for the wearer. Thus, it may be desired that a wearer-facing layer 231 not include a predominant proportion of fibers typically used as absorbers, e.g., cotton or other plant fibers, or rayon fibers. (For purposes herein, the term “rayon” includes rayon, viscose, lyocell, and any other fibers spun from reconstituted/regenerated cellulose.) Preferably, the wearer-facing layer will not include any proportion of such fibers greater than 10 percent by weight of the fabric of the layer 234, and preferably, will include no substantial proportion of such fibers. Suitable materials may include polypropylene, polyesters and polyamides (e.g., nylon). Examples of these materials, when used to spin fiber components and/or when having received suitable hydrophilizing treatment, impart the spun fibers with suitable hydrophilic surface attributes (enhancing wicking), with relatively low individual fiber texture (reducing porosity and capillarity, and therefore, absorbency of the fabric). Additional materials may be incorporated in yarn or thread components for purposes of enhancing skin feel (e.g., enhancing a slick or silky feel against the skin) and/or further affecting hydrophilicity and/or reducing absorption tendencies. In some examples, polypropylene and/or polyethylene fiber components may be included for these purposes. In some examples, resins from which constituent fibers are spun may include additives to the primary polymer components, incorporated for enhancing skin feel, adjusting hydrophilicity, reducing absorbency, etc. In some examples, polyester or nylon component resins may include an additive comprising linolenic acid, to the extent of and as described in US 2017/0369698, for purposes of enhancing elongation and skin feel attributes, while reducing material usage and cost.

Although commercially processed cotton or other commercially processed plant fiber, or semi-synthetic, cellulose-derived materials such as rayon may be considered for inclusion as component material for the absorbent layer for their inherent absorbency attributes, some of these materials may have a tendency, undesirably, to retain constituents of menstrual fluid or urine following laundering. Accordingly, one or more of polyester, polyamide and/or combinations thereof may be preferred as component resins or even the main/predominant component resins from which fiber components of yarn or thread components of fabrics for the absorbent layer 232 are formed. The constituent threads or yarns of the absorbent layer may be knitted in a manner that imparts a relatively lofty and/or densely fibrous network having a degree of void volume and capillarity making it suitable for absorbing fluid. In some examples, the knit may be a knitted terrycloth, or even a knitted/sheared terrycloth. The constituent threads or yarns may be or may include microfibers, for increased fiber surface area per unit x-y surface area or volume of the absorbent assembly.

For purposes of imparting elastic extensibility to layers present in the crotch region (particularly longitudinal stretch), it may be desired that yarn or thread components of one or more of the fabrics present include elastically extensible fibers, yarns or threads. In some examples, elastically extensible fibers, yarns or threads may be formed of or include elastane or spandex (such as LYCRA, currently available from The Lycra Company, Wilmington, Delaware), which are particularly elastically extensible and durable through a plurality of launderings, as compared to other elastic materials used to elasticize fabrics.

In other examples, one or more elastically extensible polymer film layers distinct from the fabric layer(s) in the crotch portion 130 may be included to impart elastic extensibility to the structure in the crotch portion as a whole.

An elastic polymer film layer may be formed of any suitable elastic polymer material. In some examples, an elastically extensible film layer may be formed by extrusion or other application of film resin in molten or semi-molten form onto a layer component fabric, whereby the molten resin partially penetrates the fabric and upon cooling forms a film that is partially mechanically enmeshed in and/or made integral with the fabric.

### Antimicrobial Agents

For purposes of hindering growth of microorganisms supported by components of absorbed urine or menstrual fluid, which may cause odor, it may be desired to include one or more antimicrobial agents in or among the materials present in the crotch portion 130. Any such antimicrobial agents are preferably included in a form adapted to remain in place and continue to be effective following a plurality of launderings of the pant. In some examples, an antimicrobial agent may include particles or fibers including a metal, metal alloy or metallic compound that includes one or more of copper, silver, zinc, aluminum or combinations thereof. In other examples an antimicrobial agent may include particles or fibers including carbon or a composition or compound including carbon. One or more of these materials may be included as additives to resins from which constituent fibers are spun, or may be included in compositions that are topically applied to constituent yarns, threads or fabrics following manufacture thereof. Such antimicrobial agents are preferably included in material(s) forming the absorbent layer 232 and/or the wearer-facing layer 231.

### Barrier Layer

Where menstrual fluid is absorbed by a fabric layer in the crotch portion 130 and crotch gusset 230, it may be desirable to include a barrier layer, e.g., barrier layer 233 (see, e.g., FIGS. 3A-3C) to prevent the absorbed fluid from passing from the absorbent layer to an outward-facing layer 234 or even to outer clothing. In some examples, a barrier layer 233 may be formed of or include a suitable liquid impermeable polymer film. In some examples, the film composition may be selected to have elastic extensibility, providing or complementing suitable elastic stretch attributes to the combination of layers present in the crotch portion 130 and/or crotch gusset 230. In some examples, a barrier layer 233 may be formed of or include a film formed in whole or in part of a polyurethane- or polyester-based resin. In some examples, a barrier layer 233 may be formed of or include a film that is formed by extrusion or other application of thermoplastic film resin in molten or semi-molten form directly onto the outward-facing surface of an overlying layer, such as absorbent layer 232, such that the film resin, while still molten, partially penetrates the fabric and thereby forms a liquid impermeable film that is partially mechanically enmeshed in and/or made integral with the fabric of the overlying layer. This also has the effects of consolidating these layers, which can reduce caliper in the crotch portion 130, and reducing or preventing wrinkling or bunching of the absorbent layer 232 upon elastic contraction. In a particular example, a polyurethane or polyester film may be formed by extrusion or other application of molten thermoplastic resin directly onto an outward-facing surface of a fabric that serves as or forms a component of absorbent layer 232. If the barrier layer 233 is separately formed, however, it may be desired to affixed it to the overlying layer, e.g. absorbent layer 232, via a suitable bonding mechanism, e.g., an adhesive disposed between the barrier layer and the overlying layer. A configuration that allows the barrier layer and the overlying layer to separate, so as to result in a void space between them in some circumstances, may be deemed undesirable because this may allow fluid to pool in the void space and thereby be uncontrolled and/or unmanaged by an absorbent structure, creating added risk of leakage. On the other hand, if the barrier layer 233 is not unified with an overlying fabric layer between peripheral regions of the gusset, the gusset may have enhanced pliability and stretchability since there is no binding effect resulting from integration of these layers. This latter construction may be preferred in circumstances in which, for example, management of only light menstruation or light incontinence is required, or alternatively, where the design of the garment otherwise has features that minimize the likelihood of void space between the barrier layer and the overlying absorbent layer(s).

The material selected for the barrier layer 233 may also be vapor permeable or “breathable” in that it can permit gas or water vapor to pass therethrough, while still being effectively liquid impermeable under ordinary conditions of the use contemplated herein, via a combination of having a porous structure for vapor permeability, but sufficiently small pore sizes and surfaces having low wettability (e.g. hydrophobic surfaces), for liquid impermeability. Various liquid impermeable, vapor permeable films and other materials are known and used in fields including personal hygiene and wound dressing applications. A liquid impermeable but vapor permeable barrier layer may be preferred in some circumstances for purposes of venting water vapor to improve wearer comfort and/or help avoid overhydration of the wearer's skin.

It some circumstances including those described herein (wherein, for example, the barrier layer forms a part of an edge scaling structure), it may be desired that the material(s) selected to form the barrier layer 233 be, or be processed to have, hydrophobic surfaces.

### Gusset Seaming to Outer Pant Structure

The layers forming the gusset 230, e.g., wearer-facing layer 231, absorbent layer 232 and barrier layer 233, may be joined together to form an assembly, by any suitable mechanism. In some examples, these layers may be joined about their perimeters by a suitable adhesive interposed therebetween (not specifically shown). In some examples, these layers may be joined about their perimeters by stitching. If stitching is included for this purpose, it may be desired that the stitching not penetrate barrier layer 233 or an edge sealing structure, such as edge sealing strip 235—to avoid creating liquid leakage pathways.

The following objectives are contemplated herein: Particularly about and proximate the leg opening edges 140 in the crotch portion 130 of the pant, it is deemed desirable to retain, as much as possible, elasticity and extensibility of the pant structure; to minimize material stack-up that may cause discomfort or impart or exacerbate “panty lines” visible through outer clothing; to provide for suitably snug fit that holds the absorbent gusset closely against the wearer's body and avoids gapping at the leg openings with shifting body movements; to prevent wicking and leakage of liquid from side edges of the gusset; and to avoid puncturing the barrier layer and edge sealing structure of the gusset with, e.g., stitches, which can provide leakage pathways.

Referring to FIG. 4A, these objectives are served by the configuration illustrated. Gusset 230 including absorbent assembly 238 is affixed to the outward-facing layer 234 of crotch portion 130 by a strip or tape of adhesive 236, which may be of the type that may be heat-pressed between an outward-facing surface of the gusset (in the example shown, outward-facing surface of barrier layer 233) and outward-facing layer 234. The adhesive 236 is laterally inset from the leg opening edge 140 of the pant by an adhesive inset distance AI. Preferably, adhesive 236 does not overlap, underly or overly the lower portion 235L of edge sealing strip 235 (when present; note optional alternative gusset structure shown in FIG. 3C); its laterally outermost edge is disposed either at the edge of, laterally inboard from, portion 235L. This laterally inset positioning of adhesive 236 may be imparted along the entirety of the portion of gusset 230 proximate the nearest leg opening edge 140. This positioning reduces material stack-up in with the edge scaling strip 235, and also, removes the adhesive 236 from proximity with the leg opening edge 140, thus removing any stiffening effect and/or elasticity-reducing effect that adhesive 236 may have on the structure where it is applied. In some examples adhesive inset distance AI may have a minimum value of 2 mm, more preferably 4 mm, even more preferably 7 mm and still more preferably 10 mm along the entirety of the gusset edge proximate the leg opening edge 140. Where a range within tolerances supported by particular manufacturing methods is desired, the inset distance AI along each leg edge may be, e.g., from 2 mm to 20 mm, more preferably from 4 mm to 17 mm, and even more preferably from 7 mm to 14 mm. Where desired based upon material choice, a strip of fabric finishing trim 237 may be adhered to outward-facing layer 234 by trim adhesive 237a, to provide a more finished-looking edge to the pant structure at the leg opening edge 140. Referring to FIG. 4B, in another example, adhesive 236 may extend across the gusset to a greater extent, but still be inset from the leg opening edges by distance AI as shown. No stitching is required.

FIG. 5 illustrates another example configured to serve the objectives identified above. A pattern of discrete “micro-dots” of adhesive 236d may be disposed between the gusset 230 and the outward-facing layer 234 in any desired position, including between the lower portion 235L of edge sealing strip 235, and outward-facing layer 234. Adhesive in the form of a pattern of discrete micro-dots may be applied to a substrate material layer by, e.g. SEWFREE ON DEMAND equipment available from Bemis Associates Inc., Shirley, Massachusetts, USA. Other alternatives are commercially available, including patterns of adhesive micro dots that are applied to transfer/release strips or sheets (paper or film) in a configuration enabling them to be transferred to fabric via iron or press. One such alternative is sold as PINBOND transfer adhesive, a product of New Textile Technologies GmbH, Balingen, Germany. A pattern of discrete micro-dots of adhesive may be an effective fastening mechanism for purposes herein in that fastening strength is provided through numerosity of discrete, closely-spaced fastening points at each of the micro-dots, while at the same time the micro-dots of adhesive do not have a substantial binding or stiffening effect on stretchable layer substrates to which they are adhered, because the substrates may stretch and flex between the microdots. The pattern also will typically have a substantially lower basis weight of adhesive, compared to a continuous strip or tape of adhesive. Thus, the adhesive micro-dots can be effective to adhere respective superadjacent/subjacent materials together, but also help substantially mitigate material stack up, and avoid binding, stiffening, and reducing elasticity of the substrates that they serve to adhere together. No stitching is required.

As a supplement to, complement to, or substitute for, adhesive micro-dots, the manufacturer may prefer to join materials where adhesive might otherwise be used, via thermal compression bonding or ultrasonic bonding, for example, in the form of a pattern of discrete bonds. Such bonds and a pattern thereof may be arranged and function in a manner similar to a pattern of adhesive micro-dots.

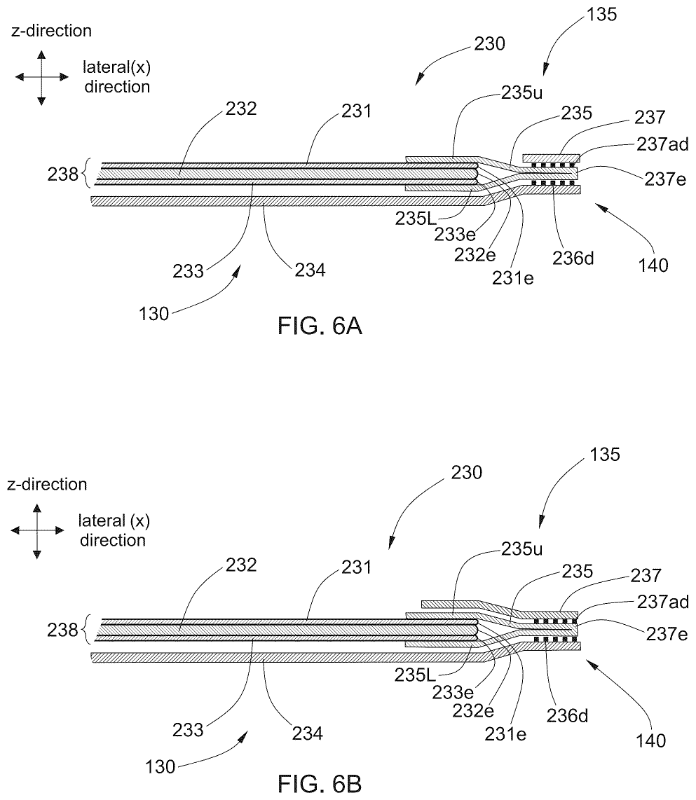

FIGS. 6A-6C illustrate additional examples configured to serve the objectives identified above. Edge sealing strip 235 is folded about itself in a “V” configuration about the peripheral edge of the absorbent assembly 238, and includes a sufficient margin of material that when pressed and adhered to itself outboard of the absorbent assembly 238 edges and proximate the fold, creates an extending portion 237e. Extending portion 237e may be attached to outward-facing layer 234 by micro-dot adhesive 236d as shown. As discussed above, the micro-dot adhesive 236d avoids material stack up, stiffening and loss of elasticity of the pant structure where it is used, in the examples shown, proximate the leg opening edges 140. These benefits may be exploited as they provide greater opportunity flexibility to include an elastically stretchable fabric trim strip 237 without substantial compromise to stretchability, elasticity and flexibility, to at least partially cover edge outer edges of sealing strip 235 and provide a finished appearance about the gusset edges. Trim strip 237 also may be adhered to the upper portion 235u of edge sealing strip by trim adhesive in micro-dot form, 237ad, as described above.

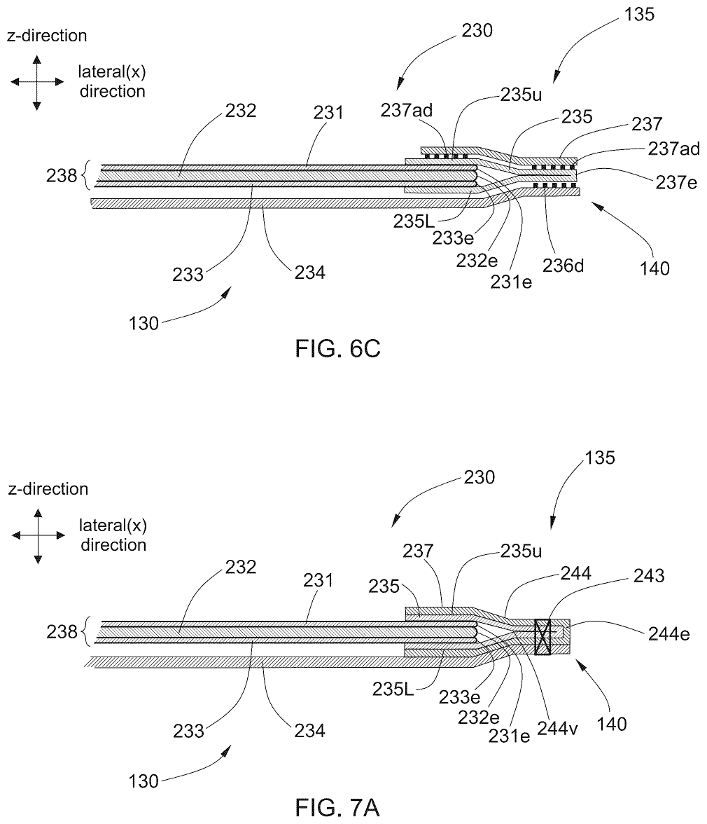

FIGS. 7A-7C illustrate additional examples configured to serve the objectives identified above. An elastic edge sealing material 235 and an extensible fabric trim material 237 may be combined as a laminate strip, which may then be folded over on itself along its length to form a combination “V”-shaped strip 244, with legs of the “V” respectively adhered to the wear-facing and outward-facing surfaces of the absorbent assembly 238. Sufficient margin may be provided outboard of the perimeter edges of assembly 238, to provide an extending flange 244c. Extending flange 244e may then be affixed to outward-facing layer 234 by any suitable mechanism, such as by stitching 243 (FIG. 7A) or micro-dot adhesive (FIG. 7B).

Where stitching is used to attach the gusset assembly 230 to the outward-facing layer 234 as suggested in FIG. 7A, the stitching is preferably of a stitch pattern that provides for extensibility along the path of the stitching. A variety of examples of potentially suitable patterns are known in the garment-manufacturing field, including, for example, zig zag stitch, lightning bolt stitch, triple straight stitch, twin needle stitch, serge stitch, blind hem stretch stitch, honeycomb stitch, feather stretch stitch, etc. It may be appreciated from FIG. 7A that, although the stitches penetrate the edge sealing material 235, the margin of extra material of the laminate 244 enables the edge sealing material 235 component thereof to be adhered to or be in sealing contact with itself inside the “V” structure, at a location 244v inboard of the stitching 243, thereby eliminating leakage pathways to the stitching 243.

In another example illustrated in FIG. 7B, the gusset 230 may be affixed to outward-facing layer 234 with micro-dot adhesive 236d, as described above.

FIG. 7C, simplified for purposes of visual clarity, illustrates another possible example, in which a scam 135 formed by stitches 243 joining gusset assembly 230 and outward-facing layer 234 together are concealed. This configuration may be formed by first stitching gusset 230 through extending flange formed of laminate 244, to outward-facing layer 234 along leg edges 14, while each are in an inverted or “inside out” configuration relative each other, while leaving the forward and rearward ends of the gusset unattached to the outward-facing layer. Next, the affixed gusset and outward-facing layer may be inverted, or turned “outside out,” to result in the configuration reflected in FIG. 7C, which internalizes and conceals the stitching 243 and presents a leg edge 140 having a hemmed and finished appearance. Although this configuration may appear to have an increased stack up of materials proximate the leg edges 140, selection of relatively low caliper, lower basis weight materials for the fabric trim material layer of laminate 244 can have a mitigating effect. Further, as can be seen, the materials forming the absorbent assembly 238 of the gusset do not underly or overlie the seam 135 joining the gusset 230 to the outward-facing layer 234 proximate the leg edges 140, and so do not contribute to material stack up at the leg opening edges. (This avoidance of stack up at the leg opening edges, by absorbent assembly 238, is generally true for all examples shown and described herein—in all examples, the perimeter edges of absorbent assembly 238, i.e., perimeter edges of layers 231, 232 and 233, are disposed laterally inboard of the leg opening edges 140.)

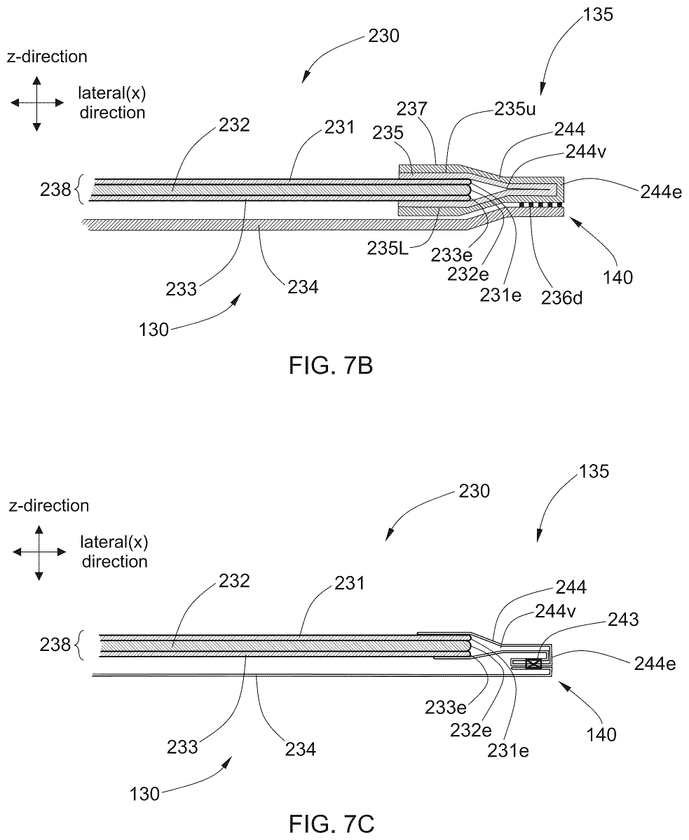

FIG. 8 reflects yet another approach. In the reflected configuration, an extra margin of outward-facing layer 234 is provided to wrap up and fold around the folded perimeter edge of edge sealing strip 235, to provide a leg opening edge 140 having a finished appearance. Deposits of micro-dot adhesive 236d may be used to adhere the gusset 230 to the outward-facing layer 234 one or more of the locations shown and/or other locations, but at least at the upper location shown, proximate the wrapped edge of outward-facing layer 234.

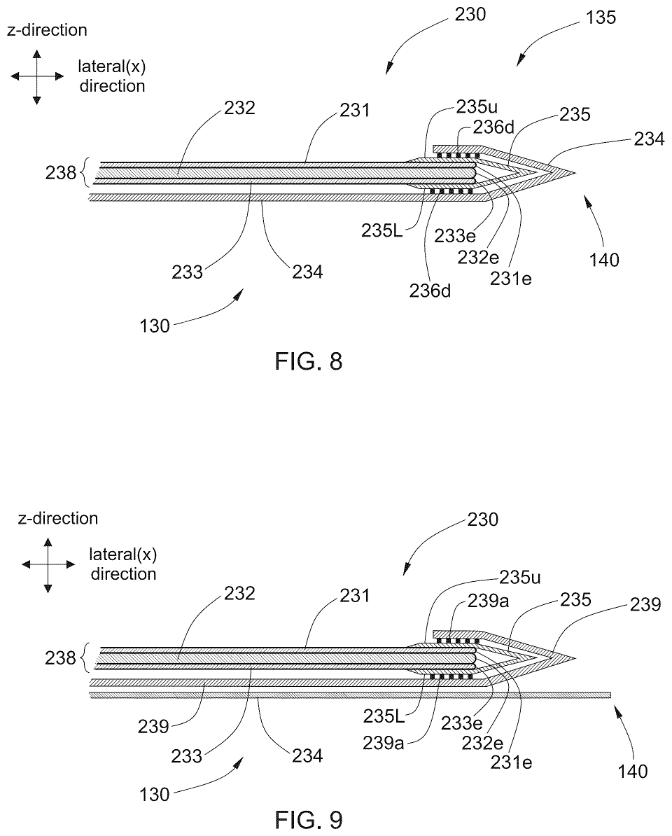

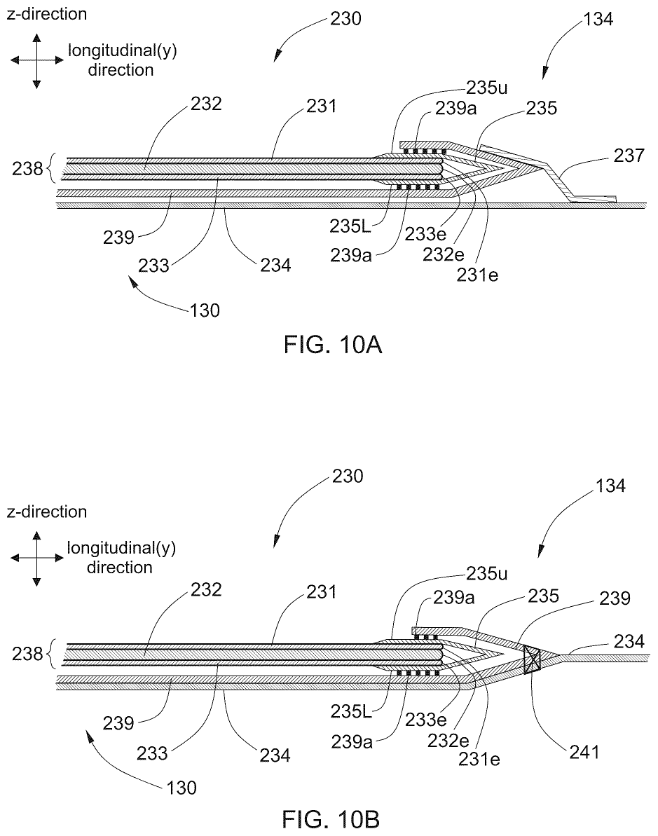

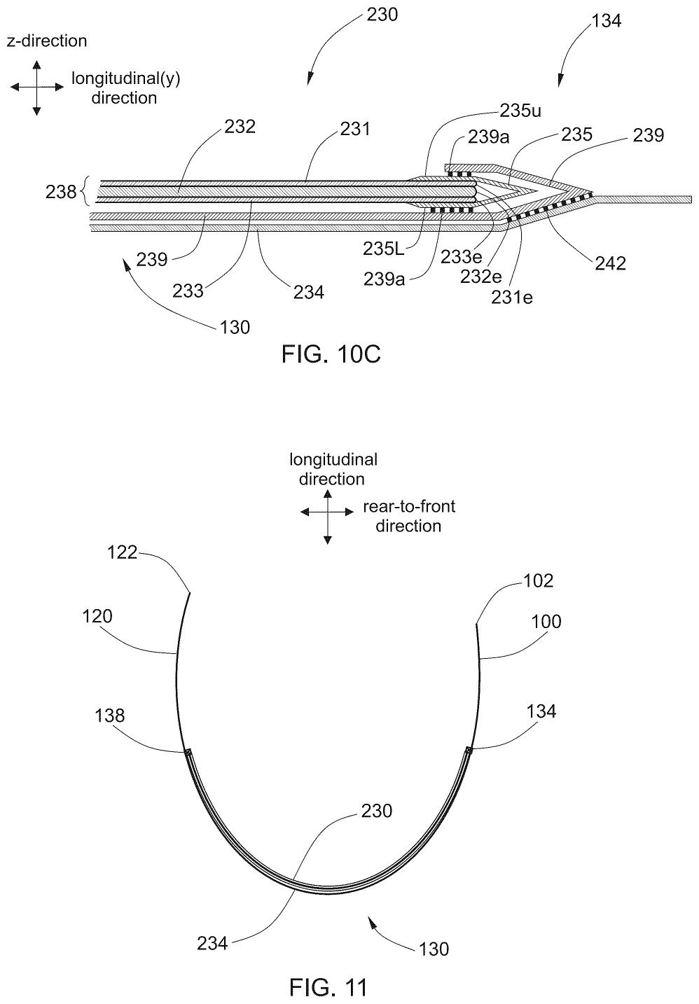

FIG. 9, viewed in conjunction with FIGS. 10A-10C and 11, reflects another approach, referred to herein as a “hammock” configuration. In this configuration, the gusset assembly 230 may be unaffixed to the outward-facing layer 234 of the pant in the crotch portion 130, and/or unaffixed to the outward-facing layer 234 along or proximate the leg edges 140, or even affixed only at forward and rearward ends, along scams 134 and 138 (shown in FIG. 2). Between the seams 134 and 138, the gusset may be unaffixed to the outward-facing layer 234. This de-coupling of the gusset 230 and outward-facing layer 234 allows the outward-facing layer 234 and any elastomeric/elastic components thereof, or affixed thereto, to move and flex independently of the components of the gusset 230, between the forward and rearward seams 134, 138, and provide better ability of the outer pant structure including outward-facing layer 234 and any associated leg band components to urge the gusset assembly against the wearer's body, particularly in configurations in which the gusset assembly might not be as stretchable/elastically extensible as the outer pant structure including outward-facing layer 234. Further, the hammock configuration enables the wearer, if she desires to wear the underpant with a supplemental sanitary napkin of the type having side “wings”, to wrap the wings over the side/leg edges of the gusset and then underneath it, between the gusset and the outward-facing layer 234, thereby concealing the wings behind the outward-facing layer. Depending upon whether the gusset assembly is more or less elastically extensible in a longitudinal direction as the underlying outer pant structure/outward-facing layer 234, it may be desired that the relaxed/contracted length of the gusset assembly between seams 134, 138 be equal to or greater than, or less than, the corresponding relaxed/contracted length of the underlying outward-facing layer(s) 234 between the seam 134, 138 locations. This may be desired to help ensure that the outer pant structure including outward-facing layer 234 is always in longitudinal tension, to help urge the gusset assembly against the wearer's body, through shifting body movements. With respect to FIG. 11, the respective lengths will be the lengths of outward-facing layer 234 and gusset assembly 230, between longitudinally innermost extents of seams 134 and 138 at the longitudinal axis 200 of the pant.

As reflected in FIG. 9, the hammock configuration may include added gusset backing layer 239, disposed beneath the barrier layer. Gusset backing layer 239 may be formed of the same fabric as outward-facing layer 234, or of a differing fabric material. Gusset backing layer 239 may be included to cover, hide and protect barrier layer 233, which in some examples may be fragile and/or may have an undesirably tacky or plastic-like feel and appearance, which the manufacturer may wish to conceal from the wearer's view and touch. In some examples, gusset backing layer 239 may be formed of a fabric that is effectively waterproof (highly resistant to passage of fluid therethrough, under conditions of use contemplated herein-like barrier layer 233 as described above). In such examples, gusset backing layer 239 may supplement or even substitute for and supplant barrier layer 233. Gusset backing layer 239 may be affixed to the absorbent assembly 238 and/or edge sealing strip 235 at the locations shown, by hammock adhesive 239a. Proximate leg edges 140, it may be desired that hammock adhesive 239a be micro-dot adhesive for the reasons explained above, while hammock adhesive 239a may be micro-dot adhesive, or tape or strip adhesive, along forward and rearward gusset seams 134, 138.

FIGS. 10A-10C schematically illustrate longitudinal cross sections of possible examples of configurations for forward and/or rearward seams 134, 138 affixing the gusset assembly 230 to the outward-facing layer 234 in the front 100 and rear 120 portions of the pant.

As reflected in FIG. 10A, a fabric trim strip 237 may be disposed to have one side overlay a forward or rearward end of the gusset 230, and a second side to overlay the outward-facing layer 234 of the pant structure. Trim strip 237 may be affixed at these locations via any suitable mechanism such as adhesive, micro-dot adhesive, stitching, etc. It will be appreciated that concerns for material stacking, binding, stiffening and loss of elasticity may not be as great for forward and rearward seams 134, 138, as for the regions of the pant proximate the leg openings, including leg opening edges 140 in the crotch portion 130.

FIGS. 10B and 10C reflect a more simplified construction, in which material of the gusset backing layer 239 is directly affixed to outward-facing layer 234, at a location that is longitudinally outboard of the edge sealing strip 123 but longitudinally inboard of the folded-over edge of the hammock layer. As reflected in FIG. 10B, the two layers of folded-over gusset backing layer 239 may be stitched with gusset end stitching 241 to the outward-facing layer 234, to form seam 134 (or for a rearward seam, 138). As reflected in FIG. 10c, the outward-facing portion of gusset backing layer 239 may be adhered to the outward-facing layer 234 along seam 134 (or for a rearward seam, 138) by a path of micro-dot gusset end adhesive 242. Noting, however, that concerns for material stacking, binding, stiffening and loss of elasticity may not be as great for forward and rearward seams 134, 138, a tape or strip adhesive may be substituted for micro-dot adhesive 242.

With respect to any of the examples depicted in FIGS. 4A through 6C, 7B, 8, 9, 10A and 10C, it will be noted that the designs reflected do not include stitched seams, but rather, rely upon adhesive, micro-dot adhesive or ultra-sonic bonding to join layers. In some examples of such designs, areas at or proximate the corners of the gusset may be vulnerable to multi-directional stresses that may precipitate separation of the material layers that are adhered or bonded, at the corners. Accordingly, referring back to FIG. 2, it may be desired to include tack stitches 245 proximate to two or all four of the gusset corners, that penetrate and join at least the outward-facing layer 234 and the gusset structure, directly, or indirectly, e.g., via/through a layer overlying wearer-facing layer 231. Such tack stitches, being discrete and localized proximate the gusset corners, will not substantially impair elastic stretch of the materials they join. Where such tack stitches are included, to avoid providing potential leakage pathways from layers to/through which fluids may wick, it may be preferred that the tack stitches do not contact and/or do not penetrate or extend through one or more of absorbent layer(s) 232 and wearer-facing layer 231. For purposes of minimizing their potential impact upon stretch or potential for causing puckering of outward-facing layer 234, such tack stitches, where included, preferably have a largest x-y plane dimension no greater than 15 mm, more preferably no greater than 12 mm, and even more preferably no greater than 10 mm, and still more preferably no greater than 8 mm.

### Other Construction Details

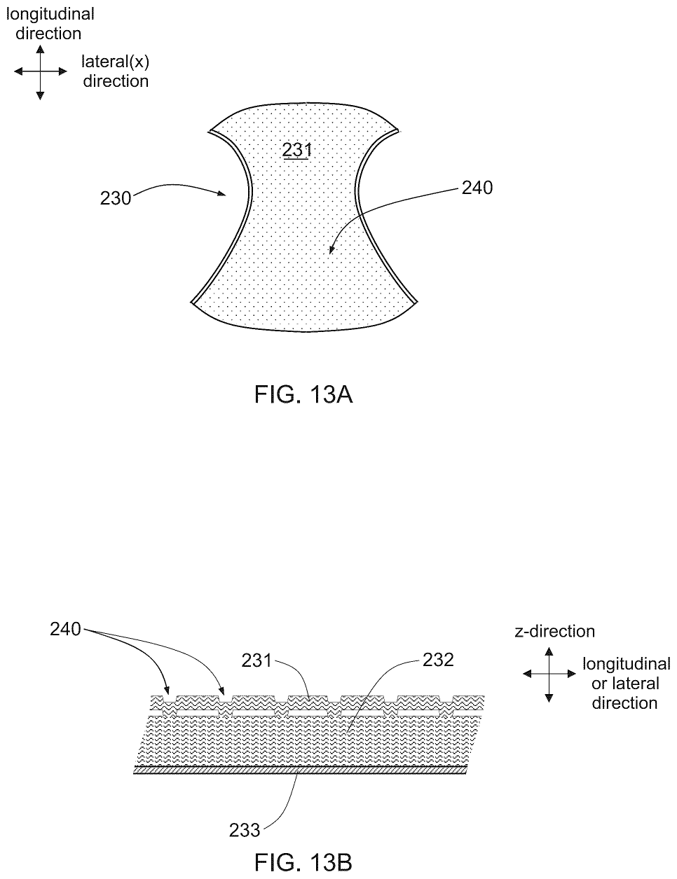

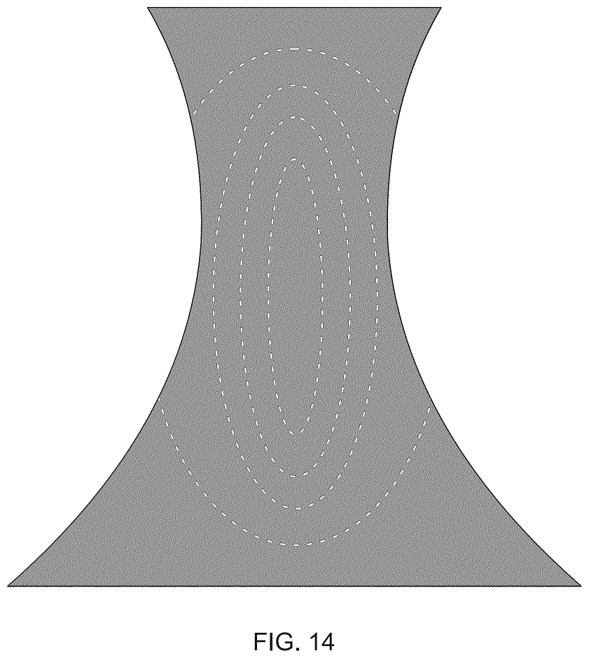

In some examples one or more layers within the crotch portion 130 and/or gusset 230 may be bonded together via any suitable bonding mechanism. Referring to FIGS. 13A and 13B for example, a wearer-facing layer 231 may be bonded to a subjacent absorbent layer 232 for purposes of holding the layers in close proximity and thereby helping maintain a desired low caliper of the structure, and enhancing fluid communication between the layers, i.e., enhancing the structure's ability to pass discharged urine or menstrual fluid from the wearer-facing layer to the underlying absorbent layer, via contact between the layers. In such example, the bonding mechanism should be selected so as not to occlude the interface between the two layers along any substantial portion of their interfacing surface areas. Thus, in some examples it may be desired that the bonding mechanism have the form of a discontinuous pattern of discrete bonds (i.e., a pattern with unbonded regions between the bonds), such as a regular pattern of spot bonds 240. In some examples, bonds between the layers may be formed by discrete deposits of adhesive between the layers, adhering them together at the locations of the deposits. In other examples, bonds between the layers may be formed by thermal compression bonding. The latter bonding mechanism may be more durable through a plurality of launderings, and as illustrated schematically in FIG. 13B, creates a corresponding pattern of z-direction depressions in the wearer-facing layer 231 that may serve to collect discharged urine/fluid and initiate and/or facilitate its movement (via wicking) down to the absorbent layer 232. For purposes of enabling thermal compression bonding, the wearer-facing layer and the subjacent absorbent layer may each include polymer components of respective fusible compositions (such as similar polyester-based compositions) that facilitate formation of robust bonds between the layers upon localized application of heat and pressure at the bond sites. (Herein, “fusible compositions,” with respect to two respective polymers present in two respective layers, means that the two polymers are miscible, capable of melting and mixing at a temperature of 250° C. or lower, to form a single thermodynamic phase.)

Various layers that may be included in crotch portion 130 and/or crotch gusset 230, e.g., layers 231, 232, 233 and 234, may also be joined to each other by any suitable mechanism at forward and rearward seams 134, 138 and crotch side seams 135 proximate the leg edges 140. The joining mechanism may be a system of stitching to affix the layers together; however, for purposes of liquid containment it may be desired that the joining mechanism include a generally hydrophobic, water insoluble adhesive or adhesive film, by itself or as a supplement to stitching, or alternatively, that such stitching be entirely above the barrier layer (not penetrating the barrier layer) or within the containment zone of the edge scaling strip 235.

### Measurement Methods

### General Sample Preparation

Unless otherwise specified below, each of the measurements below is to be conducted on 10 separate like samples (taken from 10 separate like examples of pants) and the average of the 10 separate like samples is considered to be the measurement for that specific sample set.

Samples including the entire crotch gusset 230 are collected from examples of the subject pant. Lateral lines 334, 338 that are respectively tangent the forwardmost edge of the forward seam 134 and rearwardmost edge of the rearward seam 138 are identified, and the pant is cut apart along these lines (without cutting into the seam itself) to provide a sample that includes the entire crotch gusset 230. “Crotch Width” is measured along crotch portion lateral axis 301, which is drawn along the smallest width dimension measured between the crotch leg opening edges 140.

If the pant is a legged pant, cut out the gusset in its entirely, along cutting paths outside of the seam(s) joining the gusset to the remainder of the pant, without cutting into the seams themselves. For legged pants, measurements of “Crotch Width” made for purposes herein will be the width of the removed gusset measured along the lateral direction, along a lateral line 301 marking the shortest distance between the leg openings prior to removal of the gusset from the legged pant.

The sample should be cut from the example pant with a sharp knife or suitably sharp cutting device effective to precisely and cleanly cut the sample. A straight edge or other suitable drafting/drawing tool may be used where helpful to hold the example down on the work surface and help guide the cutting device.

Samples of individual component layers from the crotch gusset, such as the wearer-facing layer 231 or absorbent layer 232 for example, are obtained by carefully cutting the seam, seal, or means of attachment around the perimeter of the crotch gusset sample. Care should be taken to not permanently deform the component layers in the separation process so as to maintain their original dimensions and the dimensions of any apertures within the layer.

All testing is performed in a room maintained at 23° C.±2° C. and 50%±2% relative humidity. Samples are conditioned for at least two hours prior to testing under the same conditions.

All linear dimensions are measured manually by ruler within the ordinary x-y plane, using a ruler that is traceable to NIST or other standards organization.

### Color Measurement Method

Color is measured on the wearer-facing surface of the absorbent layer and the wearer-facing layer of a gusset sample before and after dosing the gusset surface with artificial menstrual fluid (AMF) that has been prepared as described herein. The sample is photographed under controlled conditions. The digital photographic images are then calibrated and analyzed using image analysis software to obtain measurements of the initial (before dosing) and subsequent (after dosing) CIE 1976 L*a*b* color values. All measurements are performed at constant temperature (23° C.±2) ° C. and relative humidity (50%±2%).

The gusset sample and a calibrated color standard containing 24 standard color chips (e.g., ColorChecker Passport available from X-Rite; Grand Rapids, MI, or equivalent) are laid horizontally flat on a matte black background inside a light box that provides stable uniform lighting evenly across the entire base of the light box. A suitable light box is the GODOX LSD60 (GODOX Photo Equipment Co., Ltd., Shenzhen, China), or equivalent, which provides an inner brightness of ˜13,000 Lumens at a color temperature of ˜5800K. A digital single-lens camera with manual setting controls (e.g. a Canon EOS R available from Canon U.S.A, Inc., Melville, New York, or equivalent) is mounted directly above an opening in the top of the light box such that the entire sample and color standard are visible within the camera's field of view.

Using a standard 18% gray card (e.g., Munsell 18% Reflectance (Gray) Neutral Patch/Kodak Gray Card R-27, available from X-Rite; Grand Rapids, MI, or equivalent), the camera's white balance is custom set for the lighting conditions inside the light box. The camera's manual settings are set so that the image is properly exposed such that there is no signal clipping in any of the color channels. Suitable settings might be an aperture setting of f/8, an ISO setting of 100, and a shutter speed setting of 1/50 sec. The image is properly focused, captured, and saved as a JPEG file. The resulting image must contain the entire sample and color target at a minimum resolution of 15 pixels/mm.

### Sample Preparation:

Test samples of the wearer-facing layer and the absorbent layer from a crotch gusset are removed from the example pants as described in the General Sample Preparation section herein. Precondition samples at about 23° C.±2° C. and about 50%±2% relative humidity for 2 hours prior to testing.

### Artificial Menstrual Fluid (AMF) Preparation:

The Artificial Menstrual Fluid (AMF) is composed of a mixture of defibrinated sheep blood, a phosphate buffered saline solution and a mucous component. The AMF is prepared such that it has a viscosity between 7.15 to 8.65 centistokes at 23° C.

Viscosity of the AMF is performed using a low viscosity rotary viscometer (a suitable instrument is the Cannon LV-2020 Rotary Viscometer with UL adapter, Cannon Instrument Co., State College, PA, or equivalent). The appropriate size spindle for the viscosity range is selected, and the instrument is operated and calibrated as per the manufacturer's instructions. Measurements are taken at 23° C.±1° C. and at 60 rpm. Results are reported to the nearest 0.01 centistokes.

Reagents needed for the AMF preparation include: defibrinated sheep blood with a packed cell volume of 38% or greater (collected under sterile conditions, available from Cleveland Scientific, Inc., Bath, OH, or equivalent), gastric mucin with a viscosity target of 3-4 centistokes when prepared as a 2% aqueous solution (crude form, sterilized, available from American Laboratories, Inc., Omaha, NE, or equivalent), 10% v/v lactic acid aqueous solution, 10% w/v potassium hydroxide aqueous solution, sodium phosphate dibasic anhydrous (reagent grade), sodium chloride (reagent grade), sodium phosphate monobasic monohydrate (reagent grade) and distilled water, each available from VWR International or equivalent source.

The phosphate buffered saline solution consists of two individually prepared solutions (Solution A and Solution B). To prepare 1 L of Solution A, add 1.38±0.005 g of sodium phosphate monobasic monohydrate and 8.50±0.005 g of sodium chloride to a 1000 mL volumetric flask and add distilled water to volume. Mix thoroughly. To prepare 1 L of Solution B, add 1.42±0.005 g of sodium phosphate dibasic anhydrous and 8.50±0.005 g of sodium chloride to a 1000 mL volumetric flask and add distilled water to volume. Mix thoroughly. To prepare the phosphate buffered saline solution, add 450±10 mL of Solution B to a 1000 mL beaker and stir at low speed on a stir plate. Insert a calibrated pH probe (accurate to 0.1) into the beaker of Solution B and add enough Solution A, while stirring, to bring the pH to 7.2±0.1.

The mucous component is a mixture of the phosphate buffered saline solution, potassium hydroxide aqueous solution, gastric mucin and lactic acid aqueous solution. The amount of gastric mucin added to the mucous component directly affects the final viscosity of the prepared AMF. To determine the amount of gastric mucin needed to achieve AMF within the target viscosity range (7.15-8.65 centistokes at 23° C.) prepare 3 batches of AMF with varying amounts of gastric mucin in the mucous component, and then interpolate the exact amount needed from a concentration versus viscosity curve with a least squares linear fit through the three points. A successful range of gastric mucin is usually between 38 to 50 grams.

To prepare about 500 mL of the mucous component, add 460±10 mL of the previously prepared phosphate buffered saline solution and 7.5±0.5 mL of the 10% w/v potassium hydroxide aqueous solution to a 1000 mL heavy duty glass beaker. Place this beaker onto a stirring hot plate and while stirring, bring the temperature to 45° C.±5° C. Weigh the pre-determined amount of gastric mucin (±0.50 g) and slowly sprinkle it, without clumping, into the previously prepared liquid that has been brought to 45° C. Cover the beaker and continue mixing. Over a period of 15 minutes bring the temperature of this mixture to above 50° C. but not to exceed 80° C. Continue heating with gentle stirring for 2.5 hours while maintaining this temperature range. After the 2.5 hours has elapsed, remove the beaker from the hot plate and cool to below 40° C. Next add 1.8±0.2 mL of the 10% v/v lactic acid aqueous solution and mix thoroughly. Autoclave the mucous component mixture at 121° C. for 15 minutes and allow 5 minutes for cool down. Remove the mixture of mucous component from the autoclave and stir until the temperature reaches 23° C.±1° C.

Allow the temperature of the sheep blood and mucous component to come to 23° C.±1° C. Using a 500 mL graduated cylinder, measure the volume of the entire batch of the previously prepared mucous component and add it to a 1200 mL beaker. Add an equal volume of sheep blood to the beaker and mix thoroughly. Using the viscosity method previously described, ensure the viscosity of the AMF is between 7.15-8.65 centistokes. If not, the batch is disposed and another batch is made adjusting the mucous component as appropriate.

The qualified AMF should be refrigerated at 4° C. unless intended for immediate use. AMF may be stored in an air-tight container at 4° C. for up to 48 hours after preparation. Prior to testing, the AMF must be brought to 23° C.±1° C. Any unused portion is discarded after testing is complete.

### Image Acquisition:

Place the absorbent layer sample on the matte surface inside the light box, lay it flat with the wearer-facing surface facing upward beneath the camera along with the color standard, and capture an image of the initial wearer-facing surface of the absorbent layer. Next, place the wearer-facing layer sample on top of the absorbent layer sample in the same configuration as it was in the crotch gusset as removed from the example pant, and capture an image of the initial wearer-facing layer of the crotch gusset. Keeping the two component layers stacked together, position the tip of a mechanical pipette about 1 cm above the intersection point of the longitudinal axis and crotch portion lateral axis of the crotch gusset, and accurately pipette 1.00 mL±0.05 mL of AMF onto the surface. The fluid is dispensed without splashing, within a period of 2 seconds. After the fluid has been acquired (no pool of fluid left on the surface), a second and third dose of AMF are applied to the test sample in like fashion. After the third dose of AMF has been fully acquired, capture an image of the subsequent wearer-facing layer of the crotch gusset. Finally, carefully remove the wearer-facing layer of the crotch gusset, exposing the absorbent layer, and capture an image of the subsequent wearer-facing surface of the absorbent layer.

### Image Analysis:

To analyze an image, it is first transferred to a computer running an image analysis software (a suitable software is MATLAB, available from The Mathworks, Inc, Natick, MA, or equivalent).

The image is color calibrated using the true tristimulus XYZ color space values provided by the manufacturer for each of the 24 color chips in the color target. If target values are given in L*a*b* they are converted to XYZ according to the standard equations. The values are identified as X_{true1 . . . 24}, Y_{true1 . . . 24}, and Z_{true1 . . . 24}. Using the image analysis software the mean red, green, and blue (RGB) values of each of the 24 color chips in the image are measured using a square region of interest that covers approximately 75% of the interior area of each individual color chips. These values are identified as R_{1 . . . 24}, G_{1 . . . 24}, and B_{1 . . . 24}. A system of 24 equations, using the Xₜᵣᵤₑ and associated RGB values for each color tile, is set up according to the following example:

$${X}_{true 1} = {α}_{1} + {α}_{2} {R}_{1} + {α}_{3} {G}_{1} + {α}_{4} {B}_{1} + {α}_{5} {R}_{1}^{2} + {α}_{6} {R}_{1} {G}_{1} + {α}_{7} {G}_{1}^{2} + {α}_{8} {R}_{1} {B}_{1} + {α}_{9} {G}_{1} {B}_{1} + {α}_{10} {B}_{1}^{2}$$

$$⋮$$

$${X}_{true 24} = {α}_{1} + {α}_{2} {R}_{24} + {α}_{3} {G}_{24} + {α}_{4} {B}_{24} + {α}_{5} {R}_{24}^{2} + {α}_{6} {R}_{24} {G}_{24} + {α}_{7} {G}_{24}^{2} + {α}_{8} {R}_{24} {B}_{24} + {α}_{9} {G}_{24} {B}_{24} + {α}_{10} {B}_{24}^{2}$$

A second system of 24 equations, using the Yₜᵣᵤₑ and associated RGB values for each color tile, is set up according to the following example:

$${Y}_{true 1} = {β}_{1} + {β}_{2} {R}_{1} + {β}_{3} {G}_{1} + {β}_{4} {B}_{1} + {β}_{5} {R}_{1}^{2} + {β}_{6} {R}_{1} {G}_{1} + {β}_{7} {G}_{1}^{2} + {β}_{8} {R}_{1} {B}_{1} + {β}_{9} {G}_{1} {B}_{1} + {β}_{10} {B}_{1}^{2}$$

$$⋮$$

$${Y}_{true 24} = {β}_{1} + {β}_{2} {R}_{24} + {β}_{3} {G}_{24} + {β}_{4} {B}_{24} + {β}_{5} {R}_{24}^{2} + {β}_{6} {R}_{24} {G}_{24} + {β}_{7} {G}_{24}^{2} + {β}_{8} {R}_{24} {B}_{24} + {β}_{9} {G}_{24} {B}_{24} + {β}_{10} {B}_{24}^{2}$$

A third system of 24 equations, using the Zₜᵣᵤₑ and associated RGB values for each color tile, is set up according to the following example:

$${Z}_{true 1} = {γ}_{1} + {γ}_{2} {R}_{1} + {γ}_{3} {G}_{1} + {γ}_{4} {B}_{1} + {γ}_{5} {R}_{1}^{2} + {γ}_{6} {R}_{1} {G}_{1} + {γ}_{7} {G}_{1}^{2} + {γ}_{8} {R}_{1} {B}_{1} + {γ}_{9} {G}_{1} {B}_{1} + {γ}_{10} {B}_{1}^{2}$$

$$⋮$$

$${Z}_{true 24} = {γ}_{1} + {γ}_{2} {R}_{24} + {γ}_{3} {G}_{24} + {γ}_{4} {B}_{24} + {γ}_{5} {R}_{24}^{2} + {γ}_{6} {R}_{24} {G}_{24} + {γ}_{7} {G}_{24}^{2} + {γ}_{8} {R}_{24} {B}_{24} + {γ}_{9} {G}_{24} {B}_{24} + {γ}_{10} {B}_{24}^{2}$$

Using the 24 Xₜᵣᵤₑ equations, each of the ten α coefficients are solved for using the standard equation y=Ax, where y are the Xₜᵣᵤₑ, Yₜᵣᵤₑ, and Zₜᵣᵤₑ vectors, A is the list of the measured RGB intensities, and x is a vector of the unknown alpha (α), beta (β), or gamma (γ) coefficients to be estimated.

For example, to solve for the α's in the transform that converts the RGB colors into colorimetric X tristimulus value, the arrays are as follows:

$$\hat{x} = [ \begin{matrix}{α}_{1} \\ ⋮ \\ {α}_{10}\end{matrix} ]$$

$$A = [ \begin{matrix}1 & {R}_{1} & {G}_{1} & {B}_{1.} & {R}_{1}^{2} & … & {B}_{1}^{2} \\ ⋮ & ⋮ & ⋮ & ⋮ & ⋮ & ⋱ & ⋮ \\ 1 & {R}_{24} & {G}_{24} & {B}_{24} & {R}_{24}^{2} & … & {B}_{24}^{2}\end{matrix} ]$$

$$y = [ \begin{matrix}{X}_{true 1} \\ ⋮ \\ {X}_{true 24}\end{matrix} ]$$

The solution of the normal equations for x provides the least squares solution for the ten α coefficients according to the following equation:

\hat{x}=(A^{T}A)⁻¹A^{T}y

This procedure is repeated using the 24 Yₜᵣᵤₑ equations to solve for the ten β coefficients, and the 24 Zₜᵣᵤₑ equations to solve for the ten γ coefficients.

These coefficients are then plugged back into the original equations to provide three transform equations one each for X, Y, and Z, by which the RGB values for each individual pixel in the image are transformed into calibrated XYZ values. For example, the RGB transform equation for X using the 10 α coefficients is as follows:

$$X = {α}_{1} + {α}_{2} R + {α}_{3} G + {α}_{4} B + {α}_{5} {R}^{2} + {α}_{6} R G + {α}_{7} {G}^{2} + {α}_{8} R B + {α}_{9} G B + {α}_{10} {B}^{2}$$

The XYZ values are then converted into CIE 1976 L*a*b* values as defined in CIE 15:2004 section 8.2.1.1 using D65 reference white. Separate calibrated images are generated for each of the individual L*, a*, and b* channels for the initial and subsequent images of the wearer-facing layer of the crotch gusset and the initial and subsequent images of the wearer-facing surface of the absorbent layer.

The image resolution is calibrated using the calibrated distance scale in the image to determine the number of pixels per millimeter.

Using the image analysis software, identify a square region of interest (ROI) boundary centered at the intersection point of the longitudinal axis and crotch portion lateral axis of the crotch gusset and inscribed within the visibly discernable perimeter of the AMF stain on the image of the subsequent wearer-facing surface of the absorbent layer. Using the same ROI for each of the three channel images, the mean L*, a*, and b* value within the ROI is calculated and recorded for the subsequent wearer-facing surface of the absorbent layer to the nearest 0.1 units. The same ROI position and size is then used to measure the mean L*, a*, and b* values from the initial and subsequent images of the wearer-facing layer of the crotch gusset and the initial image of the wearer-facing surface of the absorbent layer, all of which are recorded to the nearest 0.1 units.

This entire procedure is repeated on five substantially similar replicate example pants. The reported value is the average of the five individual recorded measurements for the L*, a*, and b* values from the initial and subsequent images of the wearer-facing layer of the crotch gusset and the initial and subsequent images of the wearer-facing surface of the absorbent layer, all of which are recorded to the nearest 0.1 units.

The Delta (A) E value between two color measurements is calculated by taking the recorded average L*, a*, and b* values of a first image, identified as L*₁, a*₁, and b*₁, and the recorded average L*, a*, and b* values of a second image, identified as L*₂, a*₂, and b*₂, and then calculating the Delta (A) E value according to the following equation:

$$Delta ( Δ ) E = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}}$$

### Opacity Measurement Method

The opacity of a material is the degree to which light is blocked by that material. A higher opacity value indicates a higher degree of light block by the material. Opacity is measured using a 0° illumination/45° detection, circumferential optical geometry, spectrophotometer with a computer interface, such as the HunterLab LabScan XE running Universal Software (available from Hunter Associates Laboratory Inc., Reston, VA), or an equivalent instrument. Instrument calibration and measurements are made using the standard white and black calibration plates provided by the vendor. All testing is performed in a room maintained at about 23±2° C. and about 50±2% relative humidity.

Configure the spectrophotometer for the XYZ color scale, D65 illuminant, 10° standard observer, with UV filter set to nominal. Standardize the instrument according to the manufacturer's procedures using the 1.20 inch port size and 1.00 inch area view. After calibration, set the software to the Y opacity procedure, such that the measured opacity and XYZ color values are output.

Test samples of the wearer-facing layer and the absorbent layer from a crotch gusset are removed from the example pants as described in the General Sample Preparation section herein. Precondition samples at about 23° C.±2° C. and about 50%±2% relative humidity for 2 hours prior to testing.

Place the sample over the measurement port. The sample should completely cover the port with the surface corresponding to the wearer-facing surface of the sample directed toward the port. Cover the sample with the white standard tile. Take a reading, then remove the white tile and replace it with black standard tile without moving the sample. Obtain a second reading, and calculate the opacity as follows:

$$Opacity = Y {value}_{( black backing )} / Y {value}_{( w hite backing )} \times 100$$

A total of ten replicate samples are analyzed and their opacity results recorded. Calculate and report the average opacity to the nearest 0.1%.

Using the instrument software, convert the ten output XYZ color values of the sample backed with the white tile to the CIE 1976 L*a*b* color scale and report the average L*, a*, and b* values to the nearest 0.1 units.

The Delta (Δ) E value between two color measurements is calculated by taking the recorded average L*, a*, and b* values of a first sample, identified as L*₁, a*₁, and b*₁, and the recorded average L*, a*, and b* values of a second sample, identified as L*₂, a*₂, and b*₂, and then calculating the Delta (A) E value according to the following equation:

$$Delta ( Δ ) E = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}}$$

### % Open Area and Aperture Dimension Measurement Method

Aperture dimensions and % open area measurements are obtained from sample images acquired using a flatbed scanner. The scanner is capable of scanning in reflectance mode at a resolution of 800 dpi and 8-bit grayscale (a suitable scanner is an Epson Perfection V850 Pro from Epson America Inc., Long Beach CA, or equivalent). The scanner is interfaced with a computer running an image analysis program (a suitable program is ImageJ v. 1.53, or equivalent, National Institute of Health, USA). The acquired sample images are distance calibrated against an acquired image of a ruler certified by NIST. The resulting image is then thresheld, separating open aperture regions from sample material regions, and analyzed using the image analysis program. All testing is performed in a conditioned room maintained at about 23±2° C. and about 50±2% relative humidity.

### Sample Preparation:

Test samples of the wearer-facing layer are removed from the example pants as described in the General Sample Preparation section herein. Precondition samples at about 23° C.±2° C. and about 50%±2% relative humidity for 2 hours prior to testing.

### Image Acquisition:

Place the ruler on the scanner bed, oriented parallel to the sides of the scanner glass, and close the lid. Acquire a calibration image of the ruler in reflectance mode at a resolution of 800 dpi (approximately 31.5 pixels per mm) and 8-bit grayscale. Save the calibration image as an uncompressed TIFF format file. Lift the lid and remove the ruler. After obtaining the calibration image, all samples are scanned under the same conditions and measured based on the same calibration file. Next, place the sample onto the center of the scanner bed, lying flat, with the body-facing surface of the sample facing the scanner's glass surface. Cover the sample with a white background (in this test method white is defined as having L*>94, −2<a*<2, and −2<b*<2) and close the lid. Acquire a scanned image of the sample and save the image as an uncompressed TIFF format file. Scan and save images of the remaining nine replicates in like fashion.

### Aperture Dimension Measurements:

Open the calibration image (containing the ruler) file in the image analysis program. Perform a linear distance calibration using the imaged ruler. This distance calibration scale will be applied to all subsequent sample images prior to analysis. Open a sample image in the image analysis program. Set the distance scale. Threshold the image at a gray level value that separates the visibly identifiable boundaries between the open aperture regions from the sample material region to generate a binary image.

Using the image analysis program, analyze each of the discrete aperture regions. Measure and record all of the individual aperture areas, perimeters, feret diameters (length of the apertures), and minimum feret diameters (width of the apertures). Record the measurements for each of the individual aperture areas to the nearest 0.1 mm², the perimeters and feret diameters (length and width), to the nearest 0.1 mm. In addition to these measurements, calculate an Aspect Ratio value for each individual aperture by dividing the aperture length by its width. Repeat this analysis for each of the remaining nine replicate images. Calculate and report the statistical mean for each of the aperture dimension and the Aspect Ratio measurements using all ten of the aperture values recorded from the replicates.

### % Open Area Calculation:

Using the image analysis program, fill all of the open aperture regions in the binary image and measure the total area within the material sample region. Sum all of the individual aperture areas, divide by the total area of the sample, and multiply by 100. Record this value as the % Open Area.

In like fashion, analyze the remaining nine sample images. Calculate and report the average % Open Area value to the nearest 0.1% for the ten replicates.

### Examples

Digital photographic images of samples of wearer-facing layer material and absorbent layer material are calibrated and analyzed using image analysis software to obtain measurements of the CIE 1976 L*a*b* color values. L*a*b* color values of wearer-facing (WF) layer material samples, L*a*b* color values of absorbent layer material samples, and ΔE values between samples of wearer-facing (WF) layer material and samples of absorbent layer material are shown in Table 1.

TABLE 1

| | | | | ΔE to |
|---|---|---|---|---|
| | | | | Absorbent |
| | | | | Layer |
| Sample | L* | a* | b* | Average |
| WF blue 1 | 23.9 | 2.8 | −20.9 | |
| WF blue 2 | 23.9 | 2.9 | −21.3 | |
| Average | 23.9 | 2.9 | −21.1 | 66.3 |
| WF dark red 1 | 20.4 | 13.8 | 0.1 | |
| WF dark red 2 | 20.4 | 13.6 | −0.1 | |
| WF dark red 3 | 20.3 | 13.5 | 0.0 | |
| Average | 20.4 | 13.6 | 0.0 | 65.7 |
| WF brown 1 | 47.1 | 6.9 | 5.0 | |
| WF brown 2 | 47.2 | 6.9 | 5.1 | |
| Average | 47.1 | 6.9 | 5.0 | 38.0 |
| WF navy 1 | 22.8 | −2.7 | −13.5 | |
| WF navy 2 | 23.0 | −2.7 | −13.5 | |
| WF navy 3 | 23.2 | −2.7 | −13.4 | |
| Average | 23.0 | −2.7 | −13.5 | 64.5 |
| WF tan 1 | 60.7 | 10.4 | 15.2 | |
| WF tan 2 | 60.9 | 10.2 | 15.0 | |
| Average | 60.8 | 10.3 | 15.1 | 27.5 |
| WF gray 1 | 42.7 | 1.0 | −1.0 | |
| WF gray 2 | 42.3 | 1.1 | −1.0 | |
| Average | 42.5 | 1.0 | −1.0 | 42.5 |
| WF black 1 | 17.3 | 0.9 | −1.0 | |
| WF black 2 | 17.4 | 0.9 | −0.9 | |
| Average | 17.4 | 0.9 | −1.0 | 67.4 |
| WF black 3 | 18.1 | −0.7 | −3.1 | |
| WF black 4 | 18.1 | −0.6 | −3.1 | |
| Average | 18.2 | −0.6 | −3.1 | 66.9 |
| Absorbent | 84.6 | 0.1 | 5.5 | |
| Layer 1 | | | | |
| Absorbent | 84.5 | 0.1 | 5.7 | |
| Layer 2 | | | | |
| Average | 84.5 | 0.1 | 5.6 | |

The opacities of two samples of wearer-facing layer material are shown in Table 2.

TABLE 2

| Sample | Opacity |
|---|---|
| WF black 3 | 99.7 |
| WF black 4 | 100.4 |

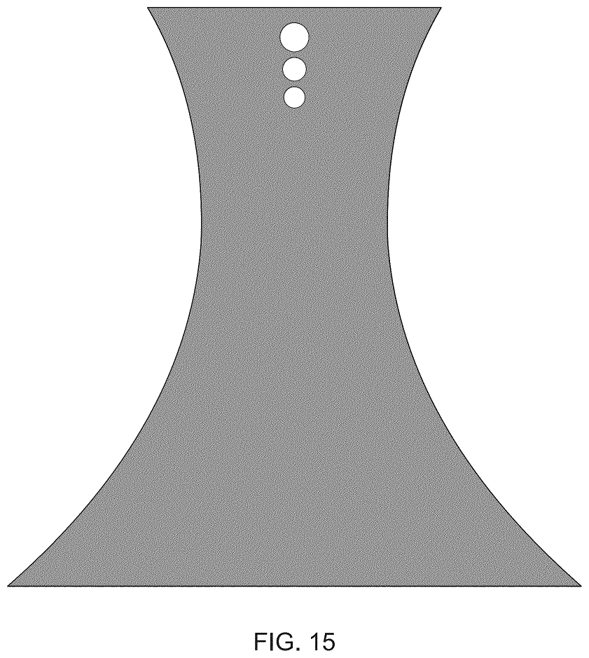

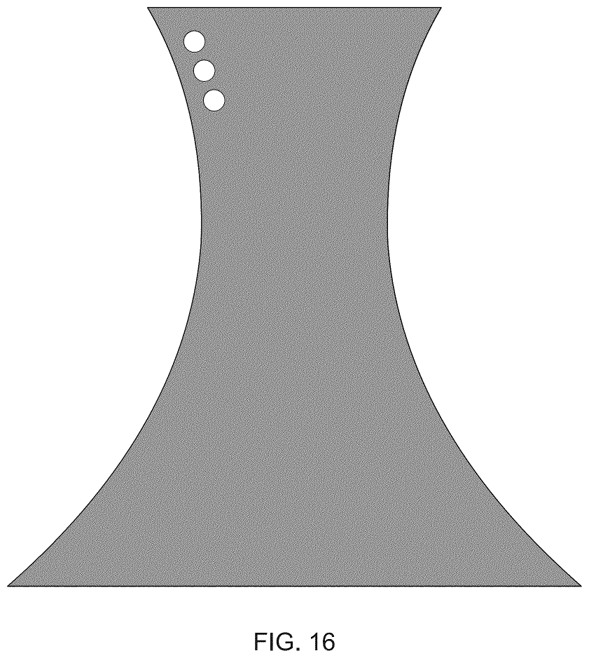

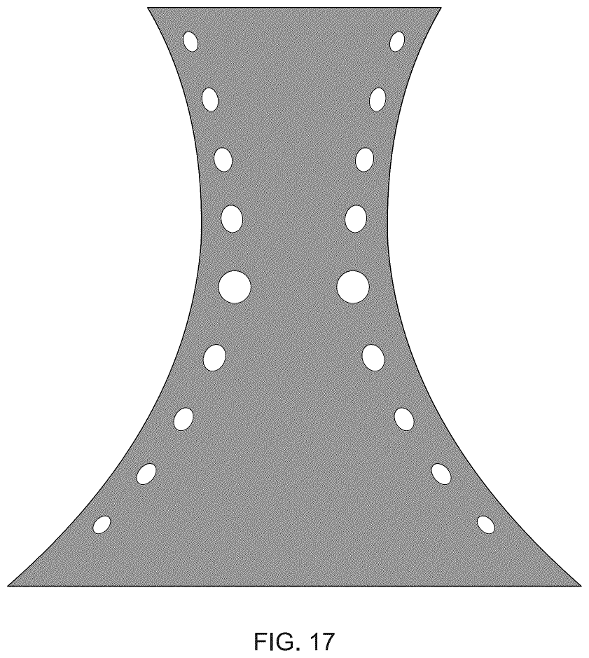

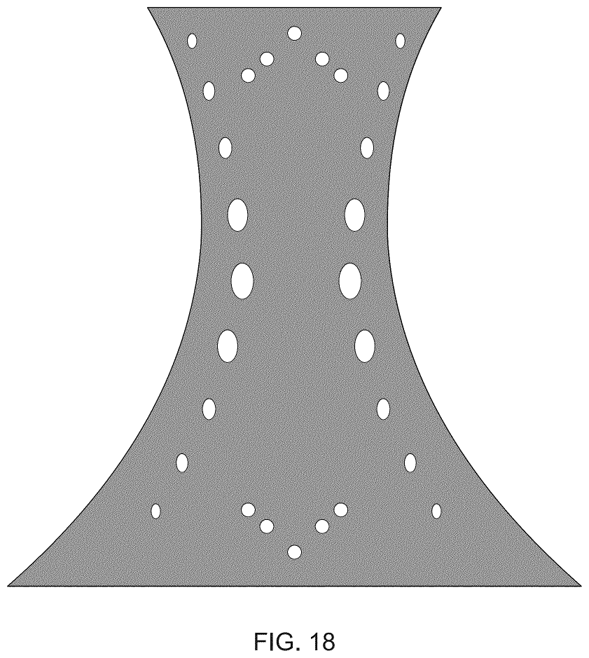

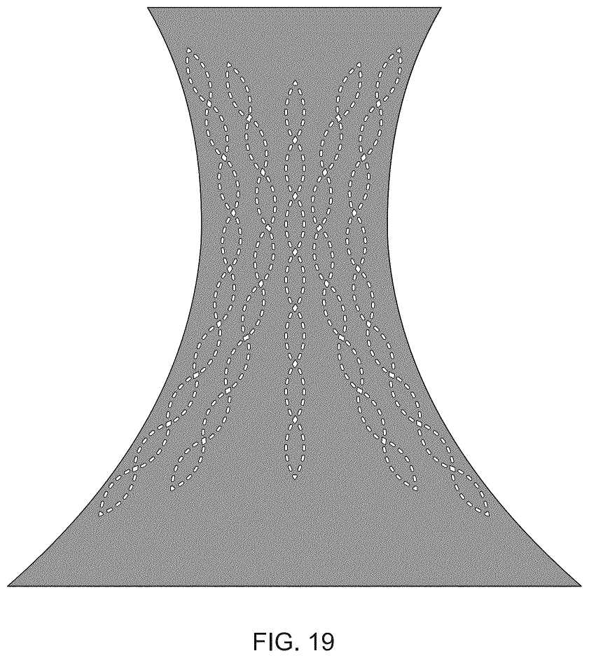

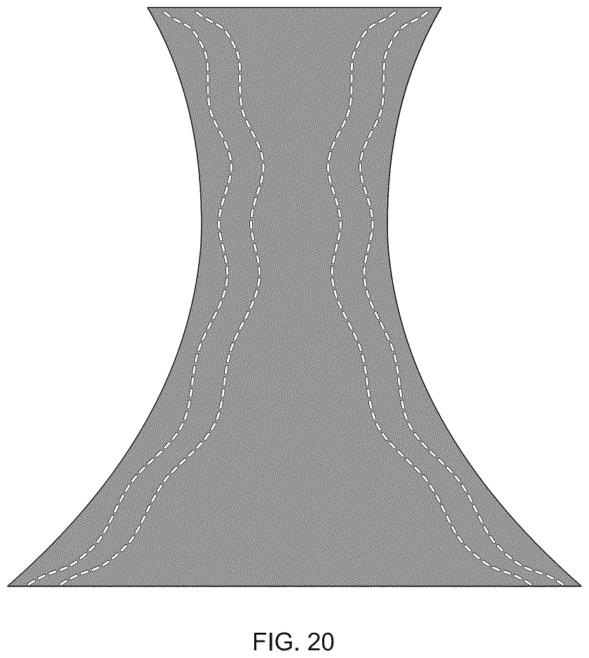

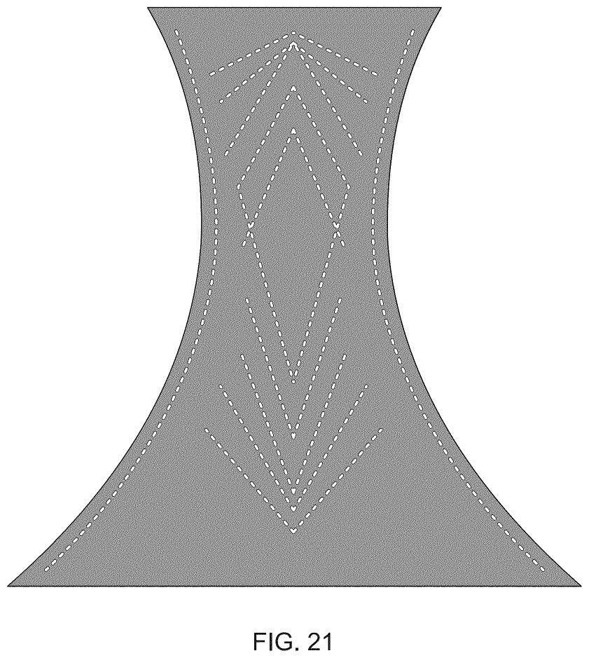

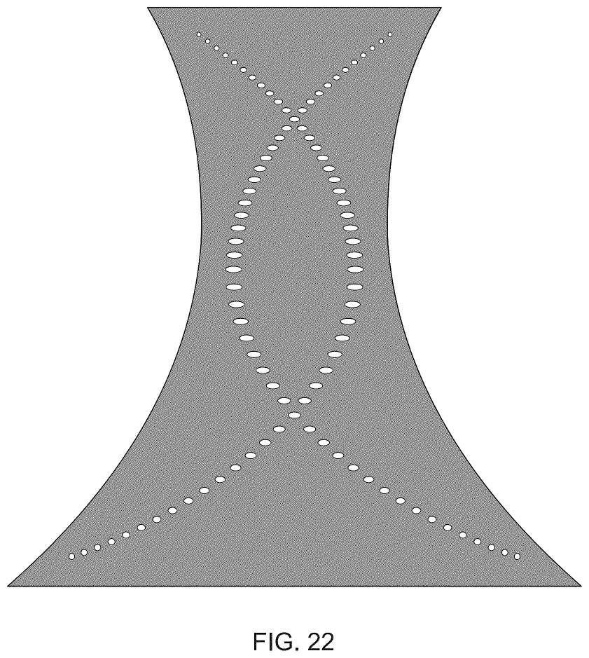

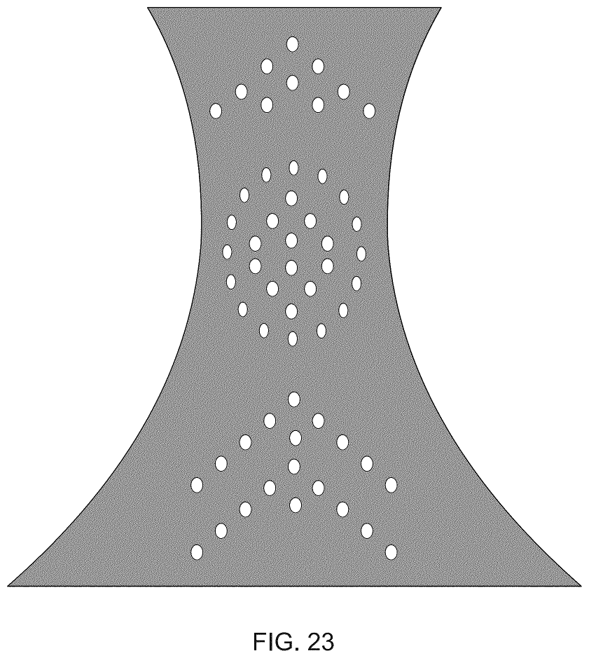

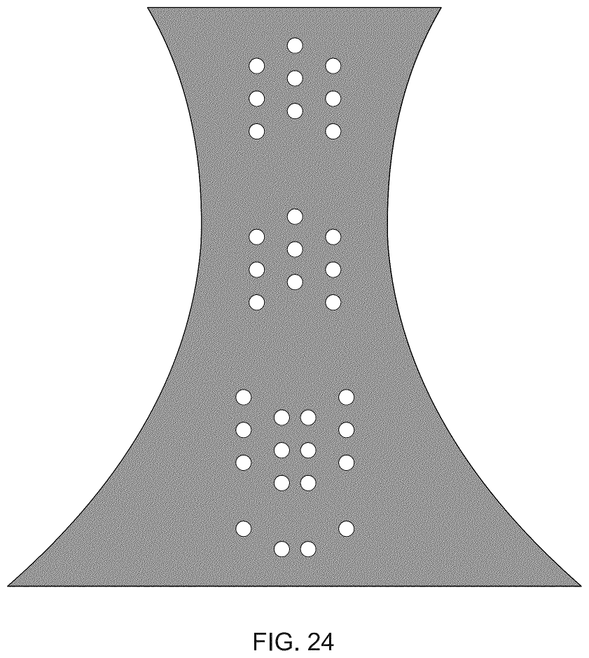

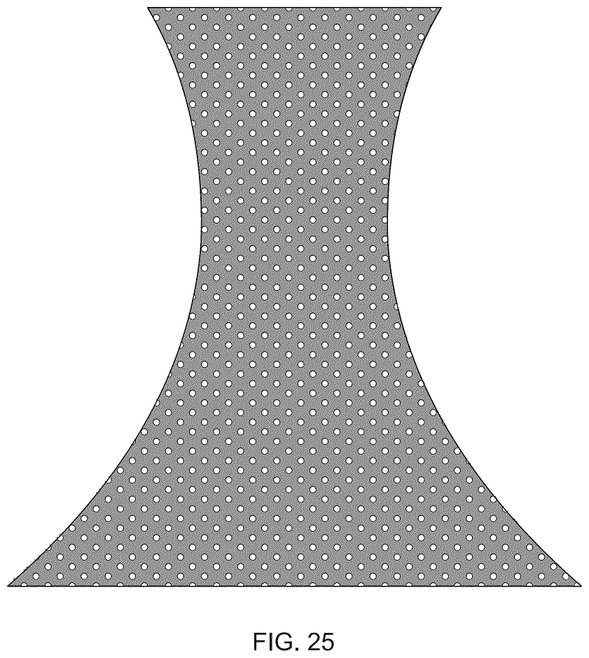

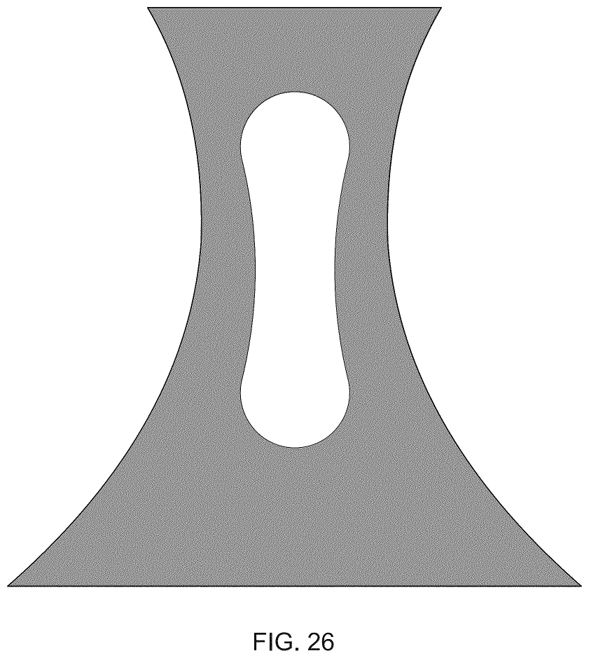

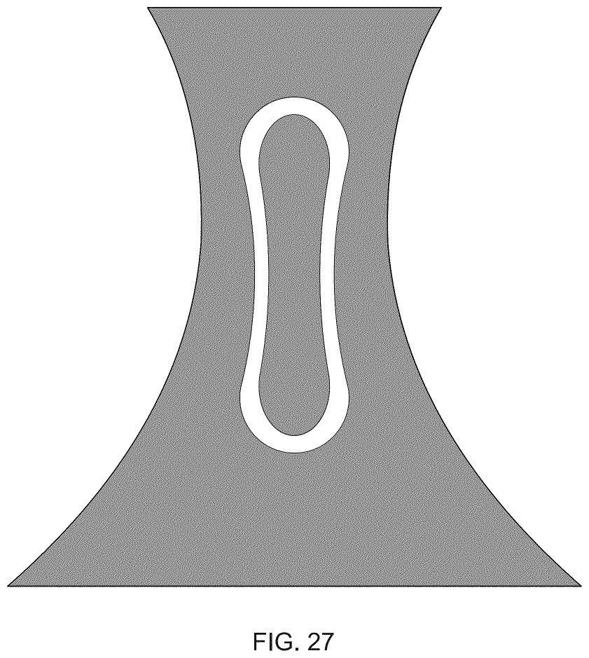

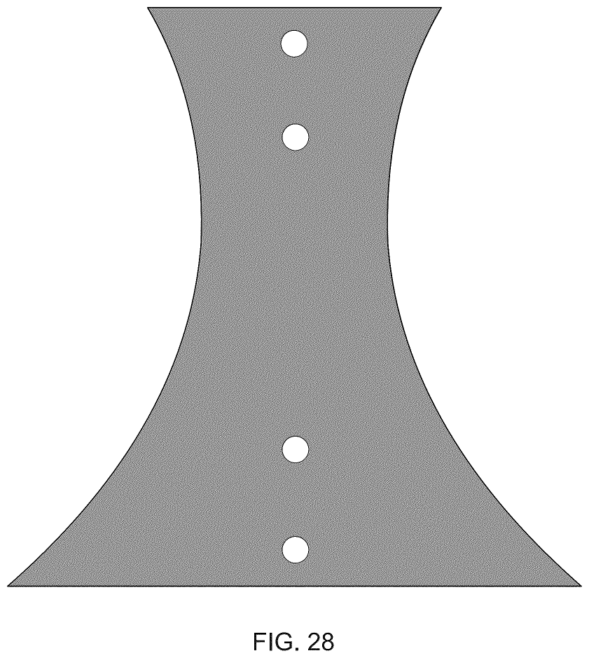

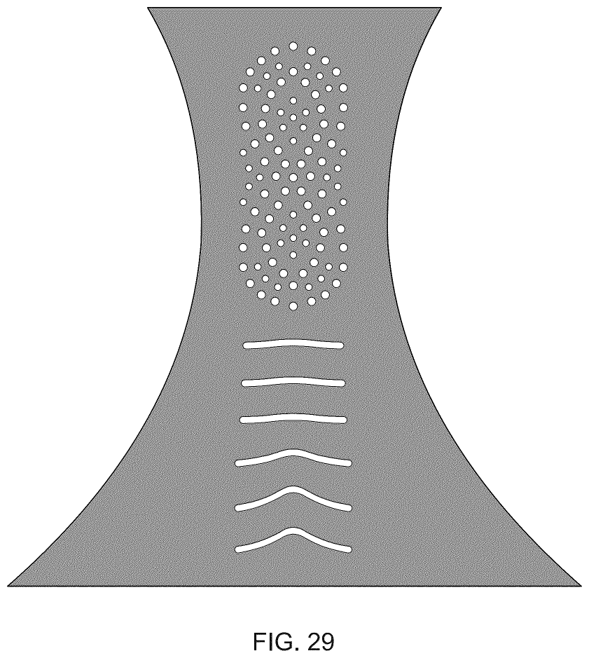

The aperture dimensions and % open area measurements of a sample image similar to FIG. 17 are shown in Table 3. The aperture dimensions and % open area measurements of a sample image similar to FIG. 15 are shown in Table 4.

TABLE 3

| | Area | Perimeter | Length | Width |
|---|---|---|---|---|
| Aperture | (mm²) | (mm) | (mm) | (mm) |
| 1 | 13.5 | 14.1 | 5.1 | 3.4 |
| 2 | 13.5 | 14.1 | 5.1 | 3.4 |
| 3 | 18.1 | 16.3 | 5.9 | 3.9 |
| 4 | 18.1 | 16.3 | 5.9 | 3.9 |
| 5 | 20.6 | 17.2 | 5.9 | 4.4 |
| 6 | 20.6 | 17.2 | 5.9 | 4.4 |
| 7 | 27.7 | 20.0 | 6.8 | 5.3 |
| 8 | 27.7 | 20.0 | 6.8 | 5.3 |
| 9 | 51.0 | 26.7 | 8.1 | 8.1 |
| 10 | 51.0 | 26.7 | 8.1 | 8.1 |
| 11 | 27.7 | 20.0 | 6.8 | 5.3 |
| 12 | 27.7 | 20.0 | 6.8 | 5.3 |
| 13 | 20.6 | 17.2 | 5.9 | 4.4 |
| 14 | 20.6 | 17.2 | 6.0 | 4.4 |
| 15 | 18.1 | 16.3 | 5.9 | 3.9 |
| 16 | 18.1 | 16.3 | 5.9 | 3.9 |
| 17 | 13.5 | 14.1 | 5.1 | 3.4 |
| 18 | 13.5 | 14.1 | 5.1 | 3.4 |
| Total Aperture Area | 421.9 | | | |
| Whole Gusset Sample | 10380.6 | 567.1 | 183.5 | 142.3 |
| Open Area % | 4.1 | | | |

TABLE 4

| | Area | Perimeter | Length | Width |
|---|---|---|---|---|
| Aperture | (mm²) | (mm) | (mm) | (mm) |
| 1 | 30.3 | 23.0 | 6.7 | 6.2 |
| 2 | 47.1 | 28.1 | 8.2 | 7.7 |
| 3 | 60.7 | 32.1 | 9.3 | 8.7 |
| Total Aperture Area | 138.1 | | | |
| Whole Gusset Sample | 16238.7 | 813.8 | 235.8 | 165.3 |
| Open Area % | 0.9 | | | |

Color is measured on the wearer-facing surface of the absorbent layer and the wearer-facing layer of a gusset sample before and after dosing the gusset surface with artificial menstrual fluid (AMF) that has been prepared as described herein. A digital photographic image of the sample is then calibrated and analyzed using image analysis software to obtain measurements of the initial (before dosing) and subsequent (after dosing) CIE 1976 L*a*b* color values. The initial (before dosing) and subsequent (after dosing) CIE 1976 L*a*b* color values are shown in Table 5.

TABLE 5

| Sample | L* | a* | b* |
|---|---|---|---|
| WF black 5 | 17.8 | 1.6 | 1.1 |
| (before dosing) | | | |
| WF black 5 | 17.1 | 1.4 | 2.1 |
| (after dosing) | | | |
| ΔE | 1.2 | | |
| Absorbent Layer 3 | 81.7 | −1.5 | 3.1 |
| (before dosing) | | | |
| Absorbent Layer 3 | 31.6 | 34.1 | 13.4 |
| (after dosing) | | | |

| | |
|---|---|
| ΔE | 62.3 |

### Ordinary X-Y Plane Dimensions

For purposes herein, when a length or width of a feature of a pant is specified, it is to be measured with the pant laid out flat on a horizontal planar surface (in an opened or assembled configuration, as appropriate) with the material of the pant smoothed out flat, but in a relaxed condition, not pulled or stretched along any planar direction.

In view of the foregoing description, the following examples are contemplated, although the identification thereof is not intended to be limiting:

A. A durable absorbent pant, comprising:
a front waist portion with a front waist edge and left and right front leg opening edges;
a rear waist portion with a rear waist edge and left and right rear leg opening edges;
a crotch portion comprising a crotch gusset, the crotch portion having a forward portion meeting the front waist portion and a rearward portion meeting the rear waist portion; and
left and right hip side portions joining the front waist portion to the rear waist portion and thereby forming a waist opening with a waist opening edge comprising the front waist edge and the rear waist edge, and left and right leg openings;
wherein, when the pant is in an opened configuration in which the front waist portion and rear waist portion are separated at the hip side portions, the pant has a longitudinal axis and a lateral axis, with an intersection thereof, the intersection occurring in the crotch portion;
wherein each of the front waist portion, rear waist portion and crotch gusset comprises a knitted material; and
wherein the crotch gusset comprises a wearer-facing layer, an absorbent layer having a length measured along the longitudinal axis, and a liquid impermeable barrier layer disposed to the outward-facing side of the absorbent layer; and
wherein the wearer-facing layer comprises a visual fullness indicator in functional association with the absorbent layer wherein the fullness indicator is configured to visibly change appearance from an initial visual state to a subsequent visual state corresponding, respectively, with absence and presence of a liquid bodily exudate in the absorbent layer or a portion thereof.

B. The durable absorbent pant according to Paragraph A, wherein the visual fullness indicator comprises one or more apertures through the wearer-facing layer.

C. The durable absorbent pant according to Paragraph B, wherein the one or more apertures have a circumscribing perimeter edge having a point location therealong, the point location being disposed a distance that is greater than about 10%, preferably greater than about 20% of the length of the absorbent layer longitudinally away from a crotch portion lateral axis.

D. The durable absorbent pant of Paragraph B, wherein the one or more apertures have a circumscribing perimeter edge having a point location therealong, the point location being disposed at about the longitudinal axis.

E. The durable absorbent pant of Paragraph B, wherein the one or more apertures have a circumscribing perimeter edge having a point location therealong, the point location being disposed a distance that is less than about 50% of a minimum width of the crotch portion laterally away from the longitudinal axis.

F. The durable absorbent pant according to any one of Paragraphs A-E, wherein a ΔL between a L* value of the wearer-facing layer and a L* value of a wearer-facing surface of the absorbent layer, according to the Opacity Measurement Method, is greater than about 5, preferably greater than about 10.

G. The durable absorbent pant according to any one of Paragraphs A-F, wherein the wearer-facing layer of the crotch gusset has a L* value of less than about 60, preferably less than about 40, according to the Opacity Measurement Method.

H. The durable absorbent pant according to any one of Paragraphs A-G, wherein the wearer-facing surface of the absorbent layer has a L* value of greater than about 50, preferably greater than about 80, according to the Opacity Measurement Method.

I. The durable absorbent pant according to any one of Paragraphs A-H, wherein a ΔE between an initial L*a*b* color value of the wearer-facing layer of the crotch gusset and a subsequent L*a*b* color value of the wearer-facing layer of the crotch gusset is less than about 30, preferably about 0.1 to about 20, more preferably about 0.5 to about 10, according to the Color Measurement Method.

J. The durable absorbent pant according to any one of Paragraphs A-I, wherein a ΔE between an initial L*a*b* color value of the wearer-facing surface of the absorbent layer and a subsequent L*a*b* color value of the wearer-facing surface of the absorbent layer is greater than about 10, preferably greater than about 50, according to the Color Measurement Method.

K. The durable absorbent pant according to any one of Paragraphs A-J, wherein a ΔE_{initial-subsequent} of the wearer-facing surface of the absorbent layer is greater than a ΔE_{initial-subsequent} of the wearer-facing layer of the crotch gusset, according to the Color Measurement Method.

L. The durable absorbent pant according to any one of Paragraphs A-K, wherein a ΔE between a subsequent L*a*b* color value of the wearer-facing layer and a subsequent L*a*b* color value of a wearer-facing surface of the absorbent layer is greater than about 10, preferably greater than about 20, according to the Color Measurement Method.

M. The durable absorbent pant according to any one of Paragraphs A-L, wherein the wearer-facing layer has an opacity of greater than about 20, preferably greater than about 25, more preferably greater than about 50, according to the Opacity Measurement Method.

N. The durable absorbent pant according to Paragraph B, wherein the one or more apertures in the wearer-facing layer of the crotch gusset together form a pattern that is symmetric about the longitudinal axis, the lateral axis, or both axes.

O. The durable absorbent pant according to Paragraph B, wherein the shape of the one or more apertures in the wearer-facing layer of the crotch gusset is a circle, an hourglass, a rectangle, a square, a diamond, a rounded diamond, a football, an oval, a clover, a heart, a triangle, a tear-drop, an ellipse, or a combination thereof, preferably a circle.

P. The durable absorbent pant according to Paragraph B, wherein the length of the one or more apertures is about 1 mm to about 20 mm.

Q. The durable absorbent pant according to Paragraph B, wherein the area of the one or more apertures is about 1 mm² to about 150 mm².

R. The durable absorbent pant according to Paragraph B, wherein the percentage of the wearer-facing layer that is apertured is about 0.1% to about 10%.

S. The durable absorbent pant according to any one of Paragraphs A-R, wherein the pant is a brief pant and has a minimum crotch width of at least about 5 cm.

T. The durable absorbent pant according to any one of Paragraphs A-S, wherein the crotch gusset exhibits a Volume Absorption Capacity of at least 0.50 ml/cm³, up to about 1.2 ml/cm³.

U. The durable absorbent pant according to any one of Paragraphs A-T, wherein the crotch portion has a central Caliper at the intersection of the lateral and longitudinal axes, preferably wherein the Caliper is no greater than 5 mm, more preferably no greater than 4 mm, and even more preferably no greater than 3.5 mm.

V. The durable absorbent pant according to any one of Paragraphs A-U, wherein the pant comprises a plurality of layer components and at least one of the layer components comprises an elastic material.

W. The durable absorbent pant according to Paragraph V, wherein the elastic material comprises one or more of elastic fiber, elastic thread, elastic yarn, or elastic film.

X. The durable absorbent pant according to any one of Paragraphs V-W, wherein the elastic material comprises a material selected from the group consisting of PET, TPEE, elastane and polyurethane, and combinations thereof.

Y. The durable absorbent pant according to any one of Paragraphs A-X, wherein the pant comprises a wearer-facing layer comprising a knitted fabric material.

Z. The durable absorbent pant according to Paragraph Y, wherein the knitted fabric material is knitted of one or more yarns that comprise synthetic material selected from the group consisting of polyester, polyamide, polypropylene, polyethylene and combinations thereof.

AA. The durable absorbent pant according to any one of Paragraphs A-Z, wherein the pant comprises an absorbent layer comprising an absorbent knitted fabric material.

BB. The durable absorbent pant according to Paragraph AA, wherein the absorbent knitted fabric material is a knitted terrycloth material.

CC. The durable absorbent pant according to any one of Paragraphs AA-BB, wherein the absorbent knitted fabric material is knitted of one or more yarns that comprise material selected from the group consisting of cotton, rayon, viscose, polyester, polyamide and combinations thereof.

DD. The durable absorbent pant according to any one of Paragraphs AA-CC, wherein the absorbent knitted fabric material comprises a microfiber yarn.

EE. The durable absorbent pant according to any one of Paragraphs A-DD, wherein the crotch portion comprises four layers comprising a wearer-facing layer, an absorbent layer directly beneath the wearer-facing layer, a liquid impermeable barrier layer beneath the absorbent layer, and an outward-facing layer beneath the barrier layer.

FF. The durable absorbent pant according to Paragraph EE, wherein any of the wearer-facing layer, absorbent layer and outward-facing layer comprises elastic filaments.

GG. The durable absorbent pant according to any one of Paragraphs EE-FF, wherein the barrier layer is a polymeric film.

HH. The durable absorbent pant according to Paragraph GG, wherein the polymeric film has been applied in liquid, semi-molten or molten form to an outward-facing side of a superadjacent knitted fabric and has thereby partially penetrated texture of the outward-facing side thereof.

II. The durable absorbent pant according to any one of Paragraphs GG-HH, wherein the polymeric film is an elastomeric film.

JJ. The durable absorbent pant according to any one of Paragraphs GG-II, wherein the polymeric film comprises a material selected from the group consisting of PET, TPEE, elastane and polyurethane, and combinations thereof.

KK. The durable absorbent pant according to any one of Paragraphs A-JJ, wherein the wearer-facing layer is thermal compression bonded to the absorbent layer in a pattern of thermal compression bonds.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A durable absorbent pant, comprising:
a front waist portion with a front waist edge and left and right front leg opening edges;
a rear waist portion with a rear waist edge and left and right rear leg opening edges;
a crotch portion joining the front waist portion and the rear waist portion;
a crotch gusset bridging the front waist portion and the rear waist portion and comprising a barrier layer, an absorbent layer overlying the barrier layer, and a wearer-facing layer overlying the absorbent layer;
left and right hip side portions joining the front waist portion to the rear waist portion and thereby forming a waist opening with a waist opening edge comprising the front waist edge and the rear waist edge, and left and right leg openings;
wherein, when the pant is in an opened configuration in which the front waist portion and rear waist portion are separated at the hip side portions, the pant has a longitudinal axis and a crotch portion lateral axis, with an intersection thereof, the intersection occurring in the crotch portion;
wherein the wearer-facing layer comprises a visual fullness indicator in functional association with the absorbent layer wherein the fullness indicator is configured to visibly change appearance from an initial visual state to a subsequent visual state corresponding, respectively, with absence and presence of a liquid bodily exudate in the absorbent layer or a portion thereof;
wherein the visual fullness indicator is distinct and located such that it has a circumscribing perimeter edge having a point location therealong, that is disposed at a distance longitudinally away from the crotch portion lateral axis by greater than 10%, of a length of the absorbent layer.

2. The durable absorbent pant of claim 1, wherein the visual fullness indicator comprises one aperture, or a distinct group of apertures, through the wearer-facing layer.

3. The durable absorbent pant of claim 2, wherein the one aperture, or distinct group of apertures, is located such that it has a circumscribing perimeter edge having a point location therealong, that is disposed at a distance longitudinally away from the crotch portion lateral axis by greater than 20%, of a length of the absorbent layer.

4. The durable absorbent pant of claim 2, wherein the one aperture, or distinct group of apertures, is not centered about the longitudinal axis.

5. The durable absorbent pant according to claim 2, wherein a ΔL between a L* value of the wearer-facing layer and a L* value of a wearer-facing surface of the absorbent layer, according to the Opacity Measurement Method, is greater than about 5.

6. The durable absorbent pant according to claim 1, wherein the wearer-facing layer of the crotch gusset has a L* value, according to the Opacity Measurement Method, of less than about 60.

7. The durable absorbent pant according to claim 1, wherein the wearer-facing surface of the absorbent layer has a L* value, according to the Opacity Measurement Method, of greater than about 50.

8. The durable absorbent pant according to claim 2, wherein a ΔE between an initial L*a*b* color value of the wearer-facing layer of the crotch gusset and a subsequent L*a*b* color value of the wearer-facing layer of the crotch gusset, according to the Color Measurement Method, is less than about 30.

9. The durable absorbent pant according to claim 2, wherein a ΔE between an initial L*a*b* color value of the wearer-facing surface of the absorbent layer and a subsequent L*a*b* color value of the wearer-facing surface of the absorbent layer, according to the Color Measurement Method, is greater than about 10.

10. The durable absorbent pant according to claim 2, wherein a ΔE_{initial-subsequent} of the wearer-facing surface of the absorbent layer is greater than a ΔE_{initial-subsequent} of the wearer-facing layer of the crotch gusset, according to the Color Measurement Method.

11. The durable absorbent pant according to claim 1, wherein a ΔE between a subsequent L*a*b* color value of the wearer-facing layer and a subsequent L*a*b* color value of a wearer-facing surface of the absorbent layer, according to the Color Measurement Method, is greater than about 10.

12. The durable absorbent pant according to claim 1, wherein the wearer-facing layer has an opacity of greater than about 20, according to the Opacity Measurement Method.

13. The durable absorbent pant according to claim 2, wherein the one or more apertures in the wearer-facing layer of the crotch gusset together form a distinct shape that is symmetric about the longitudinal axis, the lateral axis, or both axes.

14. The durable absorbent pant according to claim 2, wherein the shape of the one or more apertures in the wearer-facing layer of the crotch gusset is a circle, an hourglass, a rectangle, a square, a diamond, a rounded diamond, a football, an oval, a clover, a heart, a triangle, a tear-drop, an ellipse, a letter, a number, a symbol, or a combination thereof.

15. The durable absorbent pant according to claim 2, wherein a length of the one aperture or each of the group of apertures is about 1 mm to about 20 mm.

16. The durable absorbent pant according to claim 2, wherein an area of the one aperture or each of the group of apertures is about 1 mm² to about 150 mm².

17. The durable absorbent pant according to claim 2, wherein the percentage of the wearer-facing layer that is apertured is 0.1% to 10%.

18. A durable absorbent pant, comprising:
a front waist portion with a front waist edge and left and right front leg opening edges;
a rear waist portion with a rear waist edge and left and right rear leg opening edges;
a crotch portion joining the front waist portion and the rear waist portion;
a crotch gusset bridging the front waist portion and the rear waist portion and comprising a barrier layer, an absorbent layer overlying the barrier layer, and a wearer-facing layer overlying the absorbent layer;
left and right hip side portions joining the front waist portion to the rear waist portion and thereby forming a waist opening with a waist opening edge comprising the front waist edge and the rear waist edge, and left and right leg openings;
wherein, when the pant is in an opened configuration in which the front waist portion and rear waist portion are separated at the hip side portions, the pant has a longitudinal axis and a crotch portion lateral axis, with an intersection thereof, the intersection occurring in the crotch portion;
wherein the wearer-facing layer comprises a visual fullness indicator in functional association with the absorbent layer wherein the fullness indicator is configured to visibly change appearance from an initial visual state to a subsequent visual state corresponding, respectively, with absence and presence of a liquid bodily exudate in the absorbent layer or a portion thereof;
wherein the visual fullness indicator comprises a colorant adapted to change its appearance upon contact with menstrual fluid.

19. The durable absorbent pant of claim 18 wherein the colorant is a hydrochromic ink or coating.

20. The durable absorbent pant of claim 18 wherein the visual fullness indicator is located such that it has a circumscribing perimeter edge having a point location therealong, that is disposed at a distance longitudinally away from the crotch portion lateral axis by greater than 10%, of a length of the absorbent layer.
